(19) United States
(12) Patent Application Publication
(10) Pub. No.: US 2023/0321244 A1
(43) Pub. Date: Oct. 12, 2023

(54) PHYSIOLOGICAL SALINE SOLUTION AND METHODS FOR MAKING AND USING SAME

(71) Applicant: THE CHILDREN'S HOSPITAL OF PHILADELPHIA, Philadelphia, PA (US)

(72) Inventors: Marcus Graeme Davey, Ardmore, PA (US); Christopher Gregory, Philadelphia, PA (US)

(21) Appl. No.: 17/970,503

(22) Filed: Oct. 20, 2022

Related U.S. Application Data
(60) Provisional application No. 63/257,798, filed on Oct. 20, 2021.

(51) Int. Cl. A61K 47/02; A61K 47/12; A61K 9/08; A61G 11/00

(52) U.S. Cl. CPC A61K 47/02; A61K 47/12; A61K 9/08; A61G 11/00

(57) ABSTRACT

Physiological saline solutions (PSS) and methods for making and using same are disclosed relating to extracorporeal fetal care. In one aspect, disclosed herein is a PSS comprising: an aqueous solvent; from about 1.0 mM to about 2.0 mM calcium chloride; from about 3.0 mM to about 5.0 mM potassium chloride; from about 15.0 mM to about 20 mM sodium bicarbonate; from about 90 mM to about 110 mM sodium chloride; and from about 9 to about 13 mM sodium acetate; and optionally at least one buffering agent wherein the solution has a pH ranging from about 7.0 to about 7.4. In this or other aspects the solution has an osmolarity ranging from about 250 to about 270 mOsm.

### CROSS-REFERENCE TO RELATED APPLICATIONS

This application claims the benefit of U.S. Provision Application Ser. No. 63/257,798 filed Oct. 20, 2021, the contents of which is hereby incorporated by reference as if set forth in its entirety herein.

### TECHNICAL FIELD

The present disclosure relates generally to neonatal care, and more specifically to a physiological saline solution composition and methods for using same.

### BACKGROUND

Extreme prematurity is a leading cause of infant morbidity and mortality in the United States. Premature birth may occur due to any one of a multitude of medical reasons. Respiratory failure represents a common and challenging problem associated with extreme prematurity, as gas exchange in critically preterm neonates is impaired by structural and functional immaturity of the lungs. Even with medical advances in this field, there is still a high rate of chronic lung disease and other complications of organ immaturity in prematurely born children, particularly in fetuses born prior to 28 weeks gestation. The development of a system that could support normal fetal growth and organ maturation for even a few weeks could significantly reduce the morbidity and mortality of extreme prematurity and improve quality of life in survivors. There are shortcomings with existing mechanisms for supporting premature fetuses. Existing previous attempts to achieve adequate oxygenation of the fetus in animal models have been limited by circulatory overload and cardiac failure. The known systems suffer from unacceptable complications, such as circulatory failure and contamination.

In addition to oxygenation needs, there is a further need to provide an artificial amniotic fluid that enables the fetus to develop in an environment similar to the uterus or an extrauterine chamber. In such an environment, the fetus would swallow and “inhale” the fluid and release it into the chamber. Like a biological amniotic fluid, the artificial amniotic fluid accomplishes one or more of the following tasks: allows the developing fetus to move in the chamber thereby promoting proper bone growth; enables the lungs to develop properly; keeps a constant temperature around the fetus to prevent heat loss; and protects the fetus from external stressors.

Accordingly, systems and methods for providing extracorporeal support for a premature fetus, or fetuses (preterm or term) with inadequate respiratory gas exchange to support life, due to a spectrum of conditions/disorders, may improve viability. Also, an artificial amniotic fluid or physiological saline solution (“PSS”) that acts as a substitute for a biological amniotic fluid is needed in such systems and methods to allow the premature fetus with reduced mortality and morbidities.

### SUMMARY

At least one or more of the foregoing needs are met by an extracorporeal support system which further comprises various aspects of fetal chamber assembly systems, components, and consumables such as a physiological saline solution and methods of use and preparation of the physiological saline solution are disclosed herein. According to an aspect of the disclosure, a fetal chamber assembly configured to enclose and support a fetus therein includes a base configured to receive the fetus therein; a lid configured to removably contact the base to form a liquid-tight seal between the lid and the base; a growth chamber defined between the base and the lid, the growth chamber being configured to receive the fetus therein; and a cannulation chamber in fluid communication with the growth chamber, the cannulation chamber being configured to receive therein a cannulated umbilical cord of the fetus. The growth chamber is configured to be adjusted in size to accommodate the fetus during gestation based on the size of the fetus. The fetal chamber assembly is configured to receive a liquid comprising an artificial amniotic fluid from a storage vessel.

According to another aspect of the disclosure, disclosed herein is a physiologic saline solution (“PSS”) comprised of: an aqueous solvent; from about 1.0 mM to about 2.0 mM calcium chloride; from about 3.0 mM to about 5.0 potassium chloride; from about 15.0 mM to about 20 mM sodium bicarbonate; from about 90 mM to about 110 mM sodium chloride; and from about 9 mM to about 13 mM sodium acetate wherein the solution has a pH ranging from about 7.0 to about 7.4 and an osmolarity ranging from about 250 mOsm to about 270 mOsm.

In a further aspect, there is provided a method of preparing a physiological saline solution, comprising: dissolving sodium chloride in an aqueous solvent; dissolving sodium bicarbonate in the aqueous solvent; dissolving potassium chloride in the aqueous solvent; dissolving calcium chloride in the aqueous solvent; and adding a pH-modifying substance comprising a salt to the aqueous solvent in an amount sufficient to adjust the pH to a value of from about 7.0 to about 7.4 to provide the physiological saline solution. In this or other aspects, the method further comprises the step of introducing an additive selected from a growth factor, an antimicrobial peptide, or a combination thereof to the aqueous solvent. In this or other embodiments, the salt comprises an acetate salt, more specifically sodium acetate.

### BRIEF DESCRIPTION OF THE DRAWINGS

The present application is further understood when read in conjunction with the appended drawings. For the purpose of illustrating the subject matter, there are shown in the drawings exemplary aspects of the subject matter; however, the presently disclosed subject matter is not limited to the specific methods, devices, and systems disclosed. In the drawings:

FIG. 1 illustrates a perspective view of a fetal chamber assembly according to an aspect of this disclosure;

FIG. 2 illustrates the fetal chamber assembly of FIG. 1 showing the lid spaced from the base;

FIG. 3 illustrates a perspective view of a base of the fetal chamber assembly of FIGS. 1 and 2 according to an aspect of the disclosure;

FIG. 4 illustrates another perspective view of the base of FIG. 3;

FIG. 5 illustrates a top plan view of the base of FIGS. 3 and 4;

FIG. 6 illustrates a perspective view of the lid of the fetal chamber assembly of FIGS. 1-5 according to an aspect of the disclosure;

FIG. 7 illustrates a cross-sectional perspective view of the fetal chamber assembly of FIGS. 1-6;

FIG. 8 illustrates a perspective view of a growth chamber according to an aspect of this disclosure showing the top membrane spaced from the bottom and growth membranes;

FIG. 9 illustrates a cross-sectional perspective view of the growth chamber of FIG. 8 showing the top membrane contacting the bottom membrane;

FIG. 10 illustrates an exploded view of the bottom membrane and the growth membrane of the growth chamber of FIGS. 8 and 9;

FIG. 11 illustrates a side view of a fetal chamber assembly according to an aspect of the disclosure, showing a growth chamber having a first volume;

FIG. 12 illustrates a side view of the fetal chamber assembly of FIG. 11, showing the growth chamber having a second volume;

FIG. 13 illustrates a top view of a fetal chamber assembly, showing flow connections according to an aspect of the disclosure;

FIG. 14A illustrates a side cross-sectional view of a meconium sensor assembly according to an aspect of the disclosure;

FIG. 14B illustrates a rear cross-sectional view of the meconium sensor assembly of FIG. 22A;

FIG. 15 illustrates a schematic of a meconium sensor assembly according to another aspect of the disclosure;

FIG. 16 illustrates a top view of a fetal chamber assembly according to yet another aspect of the disclosure, showing an air removal port and air removal assembly;

FIG. 17 illustrates a front view of an air removal assembly according to an aspect of the disclosure;

FIG. 18 illustrates a perspective view of a fetal chamber assembly according to yet another aspect of the disclosure, showing air adjacent to an air removal port;

FIG. 19 illustrates a schematic view of a physiological saline solution circuit according to another aspect of the disclosure; and

FIG. 20 illustrates a front elevation view of a receptacle according to another aspect of the disclosure.

Aspects of the disclosure will now be described in detail with reference to the drawings, wherein like reference numbers refer to like elements throughout, unless specified otherwise.

### DETAILED DESCRIPTION OF ILLUSTRATIVE EMBODIMENTS

Systems disclosed in this application are configured to provide extracorporeal support to a premature neonate. Throughout this application, “fetus” and “neonate” may be used interchangeably, and it is to be understood that the descriptions herein are not limited solely to one term or the other. The term “fetus” may be used to describe both an in vivo fetus in a womb and a fetus or neonate that has been removed from the womb. The term “artificial amniotic fluid” or “physiological saline solution” or “PSS” may be used interchangeably and are not limited solely to one term or the other. These systems may provide an environment that is substantially similar to an environment the premature fetus would experience in utero. Viability of a premature fetus that is removed from the uterine environment and that is, for example, between about 23 weeks to about 24 weeks gestation, may be increased by placing the premature fetus in the disclosed system environments. According to some aspects of the disclosure, the system environment may be configured to: 1) limit exposure of the premature fetus to light; 2) limit exposure of the premature fetus to sound; 3) maintain the fetus submerged within a liquid environment; 4) maintain the premature fetus within a desired temperature range; or 5) any combination thereof.

The premature neonate may be kept in a suitable environment for a specific length of time to allow the neonate to develop. The environment is preferably as close to that of a natural womb as possible so that the neonatal development is similar to that of a fetus still in the womb. When the fetus is removed from the womb, the fetus may be placed into a fetal growth and development system that mimics, at least in part, a natural womb. The fetal system can maintain temperature, liquid, gas exchange, light exposure, physical stimulation, and other parameters that may be advantageous to fetal development. Blood vessels in the fetus may be connected to an external circulation system. The blood vessels may be cannulated by a suitable mechanism and method of cannulation, such that the fetus' blood can be moved from the fetus to the external circulation system (e.g. through a first blood vessel in the fetus), through the external circulation system, and then back to the fetus (e.g. through a second blood vessel in the fetus). The fetal system may be configured such that the fetus can remain therein for days, weeks, or months while the fetus is growing and developing. The fetal system may be disposed within, and be a part of, a larger assembly or system that maintains parameters of the chamber that are advantageous to the development of the fetus. Necessary nutrients, gases, and liquids may be delivered to the fetal chamber through connected systems, and waste may be removed from the fetal system through the one or more connected systems. Examples of fetal systems and related systems that are compatible with the PSS described herein are found in U.S. Pat. Nos. 10,085,907; 10,751,238; 10,864,131; 10,945,903; and US Publ. Nos. 2021/0161744; and 2021/0052453.

### Physiological Saline Solution (PSS)

Described herein is physiological saline solution (PSS) that is composed of elements necessary for desired fetal development and that has physical and chemical parameters that are beneficial to fetal growth. It will be understood that the liquid must be biocompatible with the fetus so as not to cause injury to the fetus when the liquid contacts the fetus. It will also be appreciated that the liquid should preferably not be corrosive or damaging to the components of the fetal chamber assembly 10 or other elements of the fluid circuit when the liquid is introduced into the fetal chamber assembly or other elements of the fluid circuit. The PSS may be controlled for various parameters, such as temperature, pressure, nutrient content, gaseous content, sterility, and/or other characteristics. In some aspects, it may be preferable that the PSS resembles, at least partly, amniotic fluid found in a natural human womb during pregnancy. In some embodiments, the PSS may include one or more gases dissolved therein.

In one aspect, there is a PSS comprising: an aqueous solvent; from about 1.0 mM to about 2.0 mM calcium chloride; from about 3.0 mM to about 5.0 mM potassium chloride; from about 15.0 mM to about 20 mM sodium bicarbonate; from about 90 mM to about 110 mM sodium chloride; from about 9 to about 13 mM acetate salt; and optionally at least one buffering agent wherein the solution has a pH ranging from about 7.0 to about 7.4. In this or other aspects the solution has an osmolarity ranging from about 250 to about 270 mOsm. Examples of an aqueous solvent include, but are not limited to, deionized water, distilled water, and or purified water.

In certain aspects, the PSS further comprises at least one additive selected from a growth factor, an antimicrobial peptide, and combinations thereof. Depending upon certain factors such as the impact of the additive on the stability of the solution and/or physiologic efficacy, the addition may be added during the manufacture of the PSS or alternatively added to the PSS within the PSS fluid circuit or other components of the extracorporeal fetus system. In this or other aspects, other additives that can be added to the PSS is at least one or more amino acids, proteins, carbohydrate, lipids, phospholipids, urea, enzymes, electrolytes, hormones, growth factor, antimicrobials, or combinations thereof to proximate the composition of human amniotic fluid and aid in the growth and development of the neonatal patient. Examples of amino acids include taurine, glutamine, arginine, ornithine, and combinations thereof. An example of a carbohydrate additive is dextrose. Examples of a growth factor additive include insulin-like growth factor I, insulin-like growth factor II, epidermal growth factor, hepatocyte growth factor, transforming growth factor alpha, transforming growth factor beta-1, erthyropoietin, granulocyte colony-stimulating factor, and combinations thereof. Examples of an antimicrobial peptide additive include: human alpha defensins 1-3, human β-defensin-1, human β-defensin-2, human β-defensin-3, human β-defensin-4, bactericidal/permeability-increasing protein, lactoferrin, cathelicidin, calprotectin, and combinations thereof.

As previously mentioned, certain additives to the PSS can contain antimicrobials or immunologics. The addition of one or more of these additives is to act as a prevention against pathogens, bacteria, fungi, protozoa, and viruses. Examples of these antimicrobial additives include, the α-defensins [HNP1-3], lactoferrin, lysozyme, bactericidal/permeability-increasing protein, calprotectin, secretory leukocyte protease inhibitor, psoriasin, and a cathelicidin.

Still further additives that can be added to the PSS as therapeutic agents include antibiotics, thyroxine, nutrients (i.e., dextrose, amino acids, and lipids), glucocorticoids, surfactants, and beta-adrenergic-receptor agonists.

In another aspect, stem cells from the amniotic fluid of the neonatal patient's mother can be obtained from the mother prior to the neonatal patient's entry into the system. The amniotic fluid can be obtained from the mother via amniocentesis or other means and can be grown in a controlled culture where the cells will divide and reproduce into a stem-cell line. These stem cells can be added to the PSS in an amount sufficient to achieve a desired therapeutic effect.

In another aspect, there is provided a method of preparing a PSS comprising: dissolving sodium chloride in an aqueous solvent; dissolving sodium bicarbonate in the aqueous solvent; dissolving potassium chloride in the aqueous solvent; dissolving calcium chloride in the aqueous solvent; and adding a pH-modifying substance to the aqueous solvent. In this or other embodiments, the pH-modifying substance comprises a salt. In this or more embodiments, the salt comprises an acetate salt such as, without limitation, sodium acetate, potassium acetate, magnesium acetate, or combinations thereof. The pH-modifying substance can be added in an amount sufficient to adjust the pH to a value of from about 7.0 and 7.4 to provide the physiological saline solution. Optionally, the step of adding a pH modifying substance further comprises adding hydrochloric acid to the aqueous solvent. In this or other aspects, the method further includes introducing an additive selected from a growth factor, an antimicrobial peptide, or a combination thereof to the aqueous solvent. This introduction step can occur prior to or during the use of the PSS in the fluid circuit. In one particular aspect, the PSS solution comprises: from about 1.0 mM to about 2.0 mM calcium chloride; from about 3.0 mM to about 5.0 mM potassium chloride; from about 15.0 mM to about 20 mM sodium bicarbonate; from about 90 mM to about 110 mM sodium chloride; and from about 9 to about 13 mM sodium acetate. In this or other aspects, the PSS has an osmolarity ranging from about 250 to about 270 mOsm. The method steps for preparing the PSS can be conducted consecutively in any combination or steps (e.g., a-b-c-d-e, a-c-b-d-e, etc.), concurrently (e.g., during at least a portion of any one or more steps), or any other order.

A method of preparing a PSS can include passing an aqueous solvent through a container containing sodium chloride. A method of preparing a PSS can include passing an aqueous solvent through a container containing sodium bicarbonate. A method of preparing a PSS can include passing an aqueous solvent through a container containing potassium chloride. A method of preparing a PSS can include passing an aqueous solvent through a container containing calcium chloride. A method of preparing a PSS can include passing an aqueous solvent through a container containing one or more of sodium chloride, sodium bicarbonate, potassium chloride, and calcium chloride. The sodium chloride, sodium bicarbonate, potassium chloride, and calcium chloride can dissolve into the aqueous solvent as the aqueous solvent passes through the container. The method can include sequentially passing an aqueous solvent through a first container containing a first substance and a second container containing a second substance. The first substance can be different than the second substance. The first substance can include at least one of sodium chloride, sodium bicarbonate, potassium chloride, and calcium chloride. The second substance can include at least one of sodium chloride, sodium bicarbonate, potassium chloride, and calcium chloride. The method can include passing the aqueous solvent through a container containing a pH-modifying substance such that the pH of the aqueous solvent is modified. The pH-modifying substance can include sodium acetate. The method can include passing the aqueous solvent through a container containing an additive selected from a growth factor, an antimicrobial peptide, or a combination thereof.

A method of preparing a PSS can include combining an aqueous solvent with a first solution. The first solution can be a fluid. Combining the aqueous solvent with the first solution can include diluting the first solution from a first concentration to a second concentration. The second concentration can be about 99% to about 90%, about 90% to about 80%, about 80% to about 70%, about 70% to about 60%, about 60% to about 50%, about 50% to about 40%, about 40% to about 30%, about 30% to about 20%, about 20% to about 10%, or about 10% to about 1% of the first concentration. The first solution can include sodium chloride. The first solution can include sodium bicarbonate. The first solution can include potassium chloride. The first solution can include calcium chloride. The first solution can include one or more of sodium chloride, sodium bicarbonate, potassium chloride, and calcium chloride.

A method of preparing a PSS can include passing an aqueous solvent through a substance generator. The substance generator can be an electrolytic cell. The substance generator can generate a first substance that is combined with the aqueous solution. The first substance can include sodium chloride. The first substance can include sodium bicarbonate. The first substance can include potassium chloride. The first substance can include calcium chloride. The first substance can include at least one of sodium chloride, sodium bicarbonate, potassium chloride, and calcium chloride.

### Extracorporeal Fetal Systems

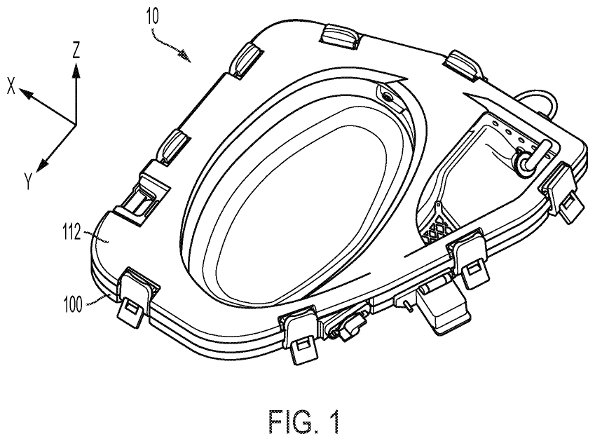

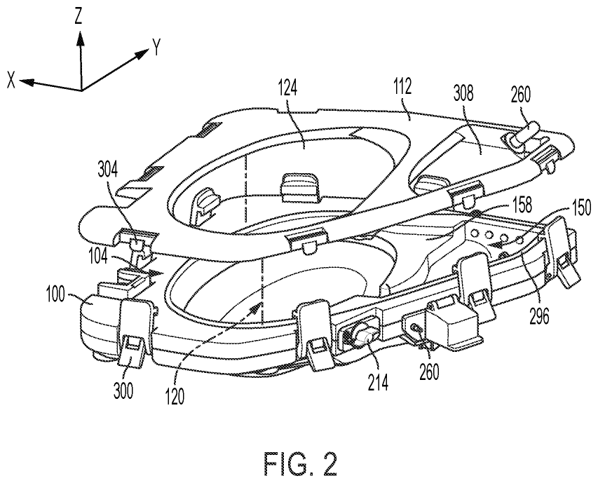

Various aspects of fetal systems and other related systems are disclosed throughout this application. In an exemplary preferred embodiment, such as that shown in FIGS. 1 and 2, a fetal chamber assembly 10 includes a base 100 and a lid 112. A growth chamber 120, which is configured to receive the fetus 1 therein, is defined in the interior space 104 between the base 100 and the lid 112. The fetus' cannulated umbilical cord 2 is disposed in a cannulation chamber 150, which has a wall structure that forms an opening into the growth chamber 120. In the preferred embodiment shown, the growth chamber 120 is configured to be adjustable in size to receive fetuses of different sizes and to accommodate growth of the fetus during the gestation period while the fetus is in the fetal chamber assembly 10. Liquid that has preferred characteristics for fetal development is introduced and flowed through the growth chamber 120 and the cannulation chamber 150. The fetus 1 may be housed in the fetal chamber assembly 10 for a desired time until it reaches a predetermined gestational stage, and the fetus 1 is monitored and maintained during the developmental process in the system 10. The fetal chamber assembly 10 may include various sensors and ports that will be described in detail below, which aid in monitoring and maintaining fetal vitals and conditions of the system 10, introducing necessities for fetal development, and removing contaminants or components of the system 10 as needed.

As shown in FIGS. 1 and 2, a fetal chamber assembly 10 includes a base 100 and a lid 112. The lid 112 may be removably affixed to the base 100, such that the lid 112 can selectively form a fluid-tight seal between the lid 112 and the base 100. The system 10 may have a closed configuration, in which the lid 112 and the base 100 form a liquid-tight seal therebetween, and an open configuration, in which a liquid-tight seal does not exist between the lid 112 and the base 100. In some aspects, the lid 112 may be entirely removable from the base 100, such that the lid 112 is not contacting, and is spaced from, the base 100. In some aspects, the lid 112 may be hingedly attached to the base 100, such that the lid 112 may pivot, along the hinged attachment, towards or away from the base 100. In some aspects, the hinged attachment (not shown) may be releasable such that the lid 112 may be entirely separated from the base 100.

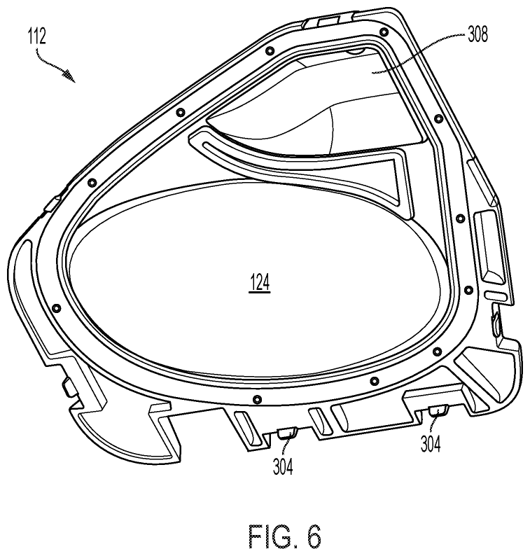

In some embodiments, the lid 112 may be configured to be affixed to the base 100 via one or more locking elements that may be selectively locked or unlocked to affix or detach, respectively, the lid 112 to or from the base 100. In some exemplary embodiments (see, e.g., FIG. 2), the base 100 may include one or more clasps 300 disposed thereon, and the lid 112 may include one or more protrusions 304 designed to be clasped by a clasp 300 disposed thereon. A different view of the lid 112 is depicted in FIG. 6. The clasps 300 on the base 100 may be configured to releasably engage with the protrusions 304 on the lid 112. It will be appreciated that other locking elements are envisioned, and this disclosure is not intended to be limited to the particular locking elements 300, 304 depicted in the figures, and that the clasps 300 can be reversed such that the clasps 300 are on the lid 112 and the protrusions 304 are on the base 100. The system 10 may include a plurality of locking elements, and the plurality of locking elements may be the same locking elements or may include different types of locking elements. Although the figures depict eight clasps 300 configured to engage with eight protrusions 304, it will be understood that another suitable number of respective base and lid closure elements can be utilized, such as 1, 2, 3, . . . 10, or another suitable number of closure elements. Easy and quick removal of the lid 112 may be beneficial in case of a medical emergency, in which a user needs to access the fetus within the interior of the fetal chamber assembly 10.

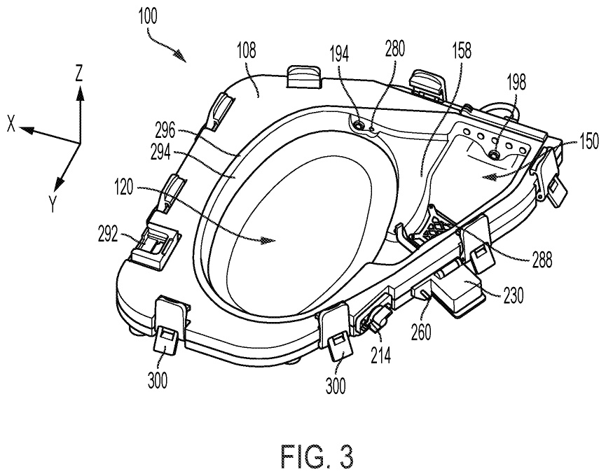

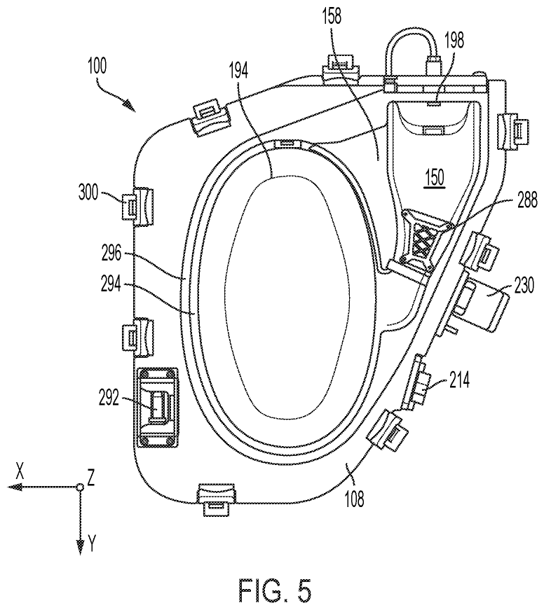

Referring to FIGS. 3-5, a base 100 is depicted according to an aspect of the disclosure. The base 100 includes a housing 108 that provides rigid structure to the base 100 and may include various ports, sensors, and channels therein, as will be described in detail later. The base 100 further includes a growth chamber 120 configured to receive the fetus therein and a cannulation chamber 150 configured to receive the fetus's cannulated umbilical cord. Suitable liquid is introduced into the system 10, for example into the housing 108, such that the liquid flows through the growth chamber 120 and through the cannulation chamber 150.

The growth chamber 120 may be surrounded, at least in part, by the housing 108. In some aspects, the growth chamber 120 may be disposed in an opening extending through the housing 108 along a vertical direction z. The growth chamber 120 may be separated from the housing by a seal 296 extending along at least a portion of the growth chamber 120. The seal 296 in the base 100 may be configured to releasably contact the lid 112 to form a liquid-tight seal between the base 100 and the lid 112. In some examples, the lid 112 may include a respective seal (not shown) configured to contact the seal 296 on the base 100. A first inlet 194, for introducing the PSS or a related liquid into the growth chamber, and an outlet 202, for discharging the PSS or related liquid from the growth chamber, are defined on the growth chamber 120. In some aspects, the first inlet 194 may be spaced away from the outlet 202 along the longitudinal direction y. It may be preferable to arrange the first inlet 194 and the outlet 202 such that the liquid enters the growth chamber 120 adjacent to the fetus's head, flows substantially along the longitudinal direction y from the fetus's head towards the fetus's feet, and exits the growth chamber 120 adjacent the fetus's feet. The arrangement of the liquid inlets and outlets will be further discussed below.

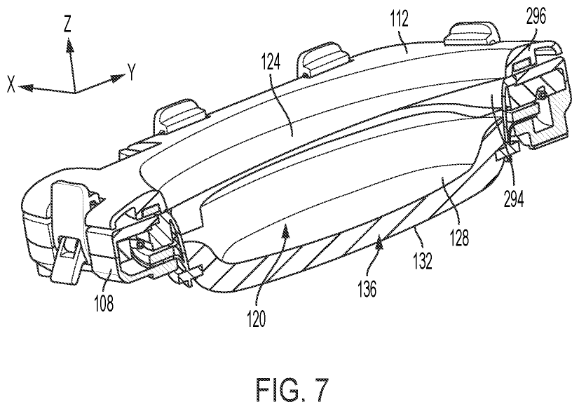

The growth chamber 120 is configured to receive and contain the fetus therein for the duration of the fetal development within the system 10. Referring to FIG. 7, which shows a cross-sectional view of the system 10 in a closed configuration, the growth chamber 120 is defined, at least in part, by a bottom membrane 128 attached to the housing 108 and by a top membrane 124 disposed in the lid 112. In some aspects, the growth chamber 120 may be further defined by the seal 296 extending circumferentially around the growth chamber 120. The seal 296 may include thereon one or more bumpers 294 that extend inward towards the growth chamber 120 and serve as physical barriers that the fetus may contact when in the growth chamber 120. The bumpers 294 are configured be soft and malleable enough so as to deform or yield when contacted by the fetus. The bumpers 294 separate the growth chamber from the rigid housing 108 and protect the fetus from incurring injury by contacting any sharp corners or rigid portions of the housing 108. The bumpers 294 may extend at least partly around the growth chamber 120. The bumpers 294 may extend between the bottom membrane 128 and the top membrane 124 along the vertical direction z. In some embodiments, the bumpers 294 may be disposed at the exterior of the growth chamber 120, such that at least one of the top membrane 124 and the bottom membrane 128 is disposed between the bumpers 294 and the fetus located inside the growth chamber 120.

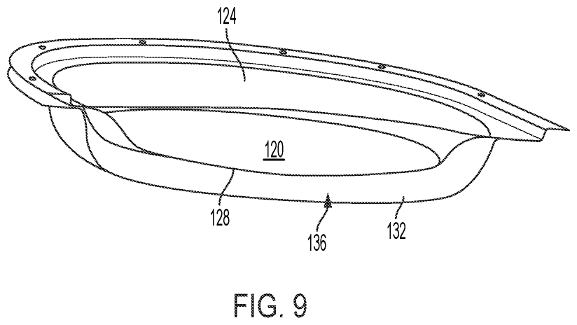

Referring generally to FIGS. 7-10, the top membrane 124 of the growth chamber 120 is spaced from the bottom membrane 128 along the vertical direction z. In practice, the fetus may be placed into the growth chamber 120, for example onto the bottom membrane 128. The growth chamber 120 is configured to receive the fetus in the space between the top membrane 124 and the bottom membrane 128. When the system 10 is moved to the closed configuration and the lid 112 is affixed to the base 100, the top membrane 124 is moved over top of the fetus and the bottom membrane 128. The top and bottom membranes 124, 128 may have the same shape or may have different shapes. For example, as shown in the figures, the bottom membrane 128 may be concave, being depressed in the vertical z direction away from the top membrane 124. The concave shape may facilitate placement of the fetus onto the bottom membrane 128. The top membrane 124 may be substantially flat in the plane defined by the transverse direction x and the longitudinal direction y. In some aspects, the top membrane 124 may be concave with the concavity extending in the vertical z direction away from the bottom membrane 128 (i.e. opposite of the concavity extending from the bottom membrane 128). The top membrane 124, the bottom membrane 128, or both membranes may be configured to stretch and extend upon application of a force, for example in the vertical z direction such that the one of, or each of, the concavities is deepened in the respective direction. In some preferred embodiments, the bottom membrane 128 may be configured to extend so as to deepen its concavity. This can increase the volume of the growth chamber 120.

### Growth Chamber

The growth chamber 120 may be configured to vary in size based on parameters of the system 10. This may be advantageous to allow the growth chamber 120 to accommodate fetuses of different sizes and also to accommodate a fetus as it grows during its residence in the system 10. In some scenarios, it is medically preferable to house the fetus in a growth chamber that is commensurate with the fetus's size. That is, it may not be preferable to receive and retain the fetus in a growth chamber that is too large. Specifically, in some aspects, it may be preferable to ensure that the fetus is not disposed in a volume that is unnecessarily large in which the fetus can be exposed to undesirable movement or getting entangled in the umbilical cord. Such entanglement may result in unwanted pressure or load to be applied to the umbilical cord, resulting in occlusion of the blood flow through the cord. It may be medically desirable to ensure that the fetus is in a small enough space that the fetus is prevented from excessive or potentially harmful shifting and repositioning within the growth chamber 120 during gestation. Such repositioning may cause injury to the fetus, strain or damage to the umbilical cord, or accidental decannulation of the umbilical cord. Conversely, it is not preferable to retain a fetus in a growth chamber that is too small for the fetus. Constricting the fetus in the growth chamber 120 may increase pressure on the fetus or hinder desired physical growth of the fetus. Controlling the fetus's positioning also helps keep the head of the fetus away from regions in the growth chamber 120 with increased risk of meconium discharge. Furthermore, controlling position of the fetus allows for various sensors and transducers to be disposed in the system 10 relative to where the fetus is expected to be positioned within the growth chamber 120. As such, it is advantageous for the system 10 to have a growth chamber 120 that can be changed in size to accommodate fetuses of varying sizes. It is further preferable to have the capability to increase the size of the growth chamber 120 to correspond to a corresponding increase in size of the fetus as the fetus grows during its residence in the system 10.

The growth chamber 120 may be configured to vary between a plurality of different volumes, with each separate volume being associated with a corresponding size of the fetus. Referring generally to FIGS. 7-12, the growth chamber 120 may have a top membrane 124 and a bottom membrane 128, as described above. The growth chamber 120 may further include a growth membrane 132 spaced away from the bottom membrane 128 generally along the vertical z direction. In some aspects, the growth membrane 132 may be disposed such that the bottom membrane 128 is arranged between the top membrane 124 and the growth membrane 132. In some preferred embodiments, the bottom membrane 128 and the growth membrane 132 may be affixed to each other along their respective perimeters, for example, by welding, heat sealing, clamping, adhesive, or another suitable fastening mechanism.

A fluid pocket 136 is defined between the bottom membrane 128 and the growth membrane 132. The fluid pocket 136 is configured to receive a fluid therein such that the fluid is retained between the bottom membrane 128 and the growth membrane 132. The fluid may include liquid and/or gas. In some preferred embodiments, the fluid is a liquid, for example, saline. In some aspects, it may be preferable for the fluid in the fluid pocket 136 to be liquid to allow diagnostic tests to be run on the growth chamber 120, such as ultrasound. It will be appreciated that the fluid may alternatively include a gas in some embodiments. The fluid inside the fluid pocket 136 is a static fluid that is not configured to contact the interior of the growth chamber 120, the fetus inside the growth chamber 120, or any liquid or components in the growth chamber 120.

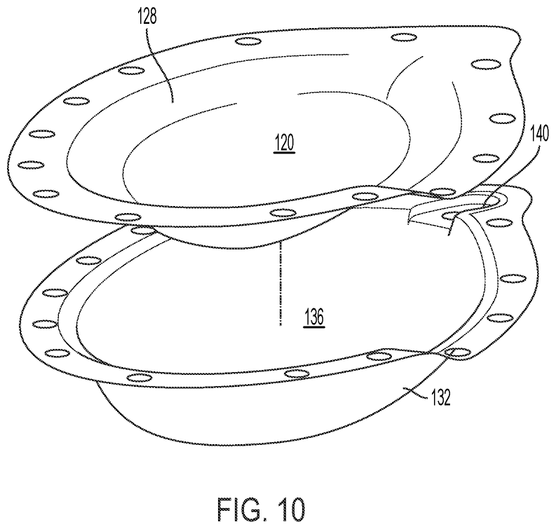

The fluid may be introduced into the fluid pocket 136 via a fluid pocket port 140 disposed on the growth chamber 120 and being in fluid communication with the fluid pocket 136 (shown in FIG. 10). In some aspects, the fluid pocket port 140 may be disposed on the bottom membrane 128. In other aspects, the fluid pocket port 140 may be disposed on the growth membrane 132. In some aspects, the fluid pocket port 140 may be disposed between the bottom membrane 128 and the growth membrane 132. The more fluid is introduced into the fluid pocket 136, the greater the volume is in the fluid pocket 136. During operation of the system 10, the fluid may be selectively added to or removed from the fluid pocket 136.

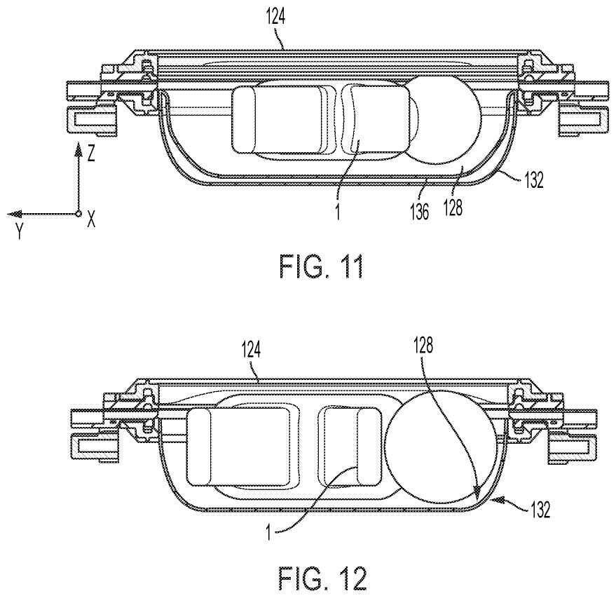

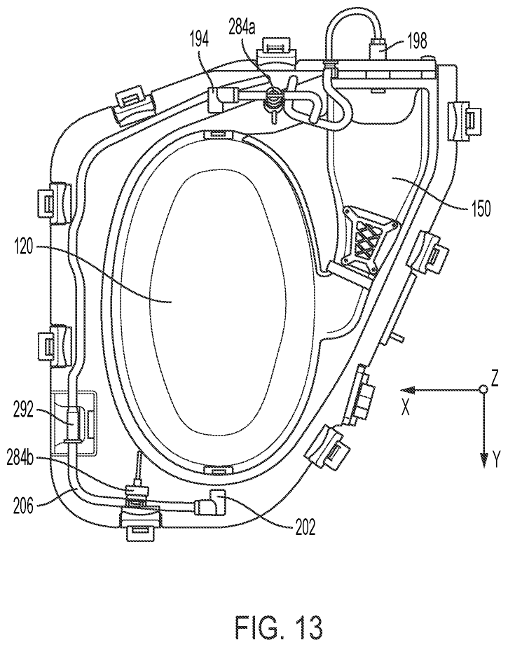

The growth chamber 120 is configured to have at least a first volume and a second volume that is different from the first volume. It will be appreciated that the growth chamber 120 may be configured to be adjusted to have any plurality of different volumes, and reference to a first or second volume is meant as a descriptive comparison of two volumes of the growth chamber 120. Referring to FIG. 11, an exemplary configuration of the growth chamber 120 is depicted having a first volume. The first volume is defined between the bottom membrane 128 and the top membrane 124. The bottom membrane 128 is spaced from the growth membrane 132 via the fluid described above. The first volume is configured to accommodate the fetus 1 having a first size. Referring to FIG. 12, an exemplary configuration of the growth chamber 120 is depicted having a second volume that is greater than the first volume. The second volume is configured to accommodate the fetus 1 having a second size that is greater than the first size. As shown in FIG. 12, the bottom membrane 128 need not be spaced from the growth membrane 132—this means that there is no fluid in the fluid pocket 136. As such, FIG. 12 depicts the largest possible volume for the embodiment of the growth chamber 120 depicted in FIGS. 11 and 12.

The specific volume of the growth chamber 120 may be inversely proportionate to the volume of the fluid pocket 136. That is, as more fluid is introduced into the fluid pocket 136, and the volume of the fluid pocket 136 is increased, the volume of the growth chamber 120 configured to receive the fetus therein is decreased. Conversely, as fluid is removed from the fluid pocket 136, and the volume of the fluid pocket 136 is decreased, the volume of the growth chamber 120 is increased. The volume of the growth chamber 120 may be defined between the top membrane 124 and the bottom membrane 128. The growth chamber 120 may be configured to change in volume along the vertical z direction, along the transverse x direction, along the longitudinal y direction, or along a combination of some or all directions. In some aspects, the volume in the growth chamber 120 may be varied in three dimensions, such that when the growth chamber volume is increased, the growth chamber 120 increases in size along the vertical z, transverse x, and longitudinal y directions, and when the growth chamber volume is decreased, the growth chamber 120 decreases in size along the vertical z, transverse x, and longitudinal y directions.

The fetus 1 may be disposed onto the bottom membrane 128, specifically on the side of the bottom membrane 128 that faces towards the top membrane 124 and that defines the volume of the growth chamber 120. The opposite side of the bottom membrane 128, which defines, in part, the fluid pocket 136, may contact the fluid in the fluid pocket 136. The fluid in the fluid pocket 136 supports the bottom membrane 128. In the aspects depicted in FIGS. 7-12, the fluid pocket 136 is arranged below the bottom membrane 128 along the vertical direction z. For purposes of this disclosure, the vertical direction z may have a non-zero vector component that is parallel to gravity. In some aspects, the vertical direction z is entirely parallel to gravity. So, the bottom membrane 128, which is disposed vertically above and is supported by the fluid in the fluid pocket 136 is being acted on by gravity along the vertical direction z, and the fluid in the fluid pocket 136 exerts a reactionary normal force on the bottom membrane 128 commensurate with the weight of the bottom membrane 128. The fetus 1, as well as any other components of the system 10, such as PSS, that are disposed on the bottom membrane 128 are similarly acted on by gravity along the vertical direction z against the fluid in the fluid pocket 136. As the amount of fluid in the fluid pocket 136 is decreased, the level of support of the bottom membrane 128 by the fluid in the fluid pocket 136 similarly decreases. As such, due to gravity, the bottom membrane 128 stretches, deforms, and/or unfolds to expand, along the transverse x and/or longitudinal y directions, and sag farther down, along the vertical direction z, towards the fluid pocket 136. As the bottom membrane 128 moves downward along the vertical direction z away from the top membrane 124, the volume inside the growth chamber 120 increases. Conversely, if the amount of fluid in the fluid pocket 136 is increased, the level of support of the bottom membrane 128 similarly increases, and the bottom membrane is propped upward along the vertical direction z towards the top membrane 124, which, in turn, decreases the volume of the growth chamber 120 defined between the top and bottom membranes 124, 128. In one embodiment, therefore, the bottom and growth membranes 128, 132 function as a variable-volume bladder mechanism.

In operation, when the fetus 1 is introduced into the growth chamber 120, the fetus 1 has a first size, and the growth chamber 120 has a first volume. The fetus 1 may be introduced onto the bottom membrane 128 along with the PSS and any other constituents of the system 10. The fluid pocket 136 may include a first amount of fluid therein that is configured to provide support to the bottom membrane 128 that opposes gravity and that is commensurate with the weight of the fetus 1, the bottom membrane 128, the PSS, and any other components contacting the bottom membrane 128 inside the growth chamber 120. As the fetus 1 grows to a second size, it may be desirable to increase the volume of the growth chamber 120 by a corresponding amount relative to the growth of the fetus 1. To do this, fluid may be removed from the fluid pocket 136 via the fluid pocket port 140 such that the fluid pocket 136 contains a second amount of fluid therein that is less than the first amount. The decrease in fluid and the physical support provided by the fluid causes the bottom membrane 128 to expand in the one or more transverse x, longitudinal y, and vertical z directions, thus increasing the volume of the growth chamber 120 to a second volume.

The process of adjusting the volume in the growth chamber 120 may be manual or automatic. In some aspects, a user (e.g. doctor or nurse) may selectively introduce or remove fluid into or out of the fluid pocket 136 in order to vary the volume inside the growth chamber 120. In some aspects, a controller and a processor may be configured to communicate with the system 10 in order to automatically add or remove fluid into or out of the fluid pocket 136. The volume adjustment process may be done based on the weight, positioning, age, health condition, or another parameter of the fetus 1. In some aspects, the volume adjustment may be done based on a particular timeline, for example, daily, bidaily, weekly, biweekly, monthly, or the like. In some aspects, the weight of the fetus 1 may be estimated using derived formulas associated with ultrasound measurements of the fetus 1 inside the growth chamber 120.

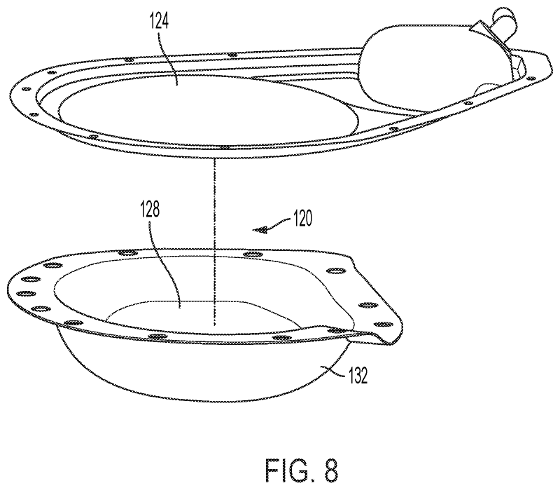

The top, bottom, and growth membranes 124, 128, 132 may include polyurethanes, polypropylenes, polyethylenes, acrylics, polyvinyl chloride, ethylene vinyl acetate, polyvinylidene chloride, or other plastics or laminated combinations of plastics. In some aspects, the top membrane 124, the bottom membrane 128, the growth membrane 132, two of the above, or all of the above, could include thermoplastic urethanes. In some aspects, the top, bottom, and growth membranes 124, 128, 132 may all include the same materials, or, alternatively, they may be composed of different materials. In some aspects, the thickness of each membrane above may be the same, or, alternatively, thicknesses may vary between at least two of the above membranes. In some specific embodiments, the growth membrane 132 may be thicker than the top membrane 124, the bottom membrane 128, or both. In some embodiments, the growth membrane 132 may be approximately twice as thick as the top membrane 124 and/or the bottom membrane 128. In some aspects, the top, bottom, and/or growth membranes 124, 128, 132 may have a durometer of between about 50 and about 100, between about 60 and about 90, between about 70 and about 80, or in a range overlapping one or more of the above ranges. In some aspects, the membranes 124, 128, and/or 132 may be formed to have a specific shape (see, e.g., FIGS. 8-10). In some aspects, it may be advantageous for the top membrane 124, the bottom membrane 128, and/or the growth membrane 132 to be transparent. In some aspects, it may be advantageous for the top membrane 124, the bottom membrane 128, and/or the growth membrane 132 to be sonolucent, such that ultrasound waves may be permitted to pass therethrough without unwanted interference or echoes.

It will be appreciated that at least the surfaces of the top membrane 124 and the bottom membrane 128 that face each other, define the growth chamber 120, and are configured to contact the fetus 1 are composed of biocompatible materials that are suitable for continued exposure to the fetus 1 and the components of the system 10 in the growth chamber (e.g. the PSS). In some aspects, it may be advantageous to ensure that at least the top and bottom membranes 124, 128 (specifically, at least, the respective surfaces disposed in the interior of the growth chamber 120) to be substantially smooth and devoid of textures or roughness that could otherwise promote bacterial growth thereon.

The particular size, shape, and dimensions of the growth chamber 120 will depend on the intended use, the size of the fetus, and manufacturing constraints. In some exemplary embodiments, the growth chamber 120 may have a first dimension measured along the longitudinal direction y of between about 3 inches and about 20 inches; between about 7 inches and about 16 inches; between about 10 inches and about 12 inches; or in another suitable range. The growth chamber 120 may have a second dimension measured along the transverse direction x of between about 3 inches and about 14 inches; between about 5 inches and about 12 inches; between about 7 inches and about 10 inches; or in another suitable range. The growth chamber 120 may have a third dimension measured along the vertical y direction of between about 2 inches and about 12 inches; between about 4 inches and about 8 inches; or in another suitable range.

### Flow Path Through Fetal Chamber Assembly

In operation, the fetal chamber assembly 10 is configured to receive a suitable liquid therein to flow through the growth chamber 120 and the cannulation chamber 150. The liquid may contact the fetus inside the growth chamber 120 and the fetus's umbilical cord inside the cannulation chamber 150 and in the growth chamber 120.

The PSS is introduced into the fetal chamber assembly 10 from a PSS source. In some aspects, it may be preferred that the PSS does not remain inside the fetal chamber assembly 10 in a stagnant state, and is instead moved at an advantageous flow rate. Avoiding stagnant liquid may help prevent bacterial growth inside the fetal chamber assembly 10. The fetal chamber assembly 10 may be configured to pass the PSS therethrough, such that new, or fresh, PSS enters the fetal chamber assembly 10, moves therethrough, and then exits the fetal chamber assembly 10, rather than continuously cycle the same PSS in a closed loop within the fetal chamber assembly 10. Introducing new PSS instead of cycling the same PSS may help prevent bacterial growth and buildup, help remove contaminants from the fetal chamber assembly 10, and may provide better gas and nutrient exchange for fetal development.

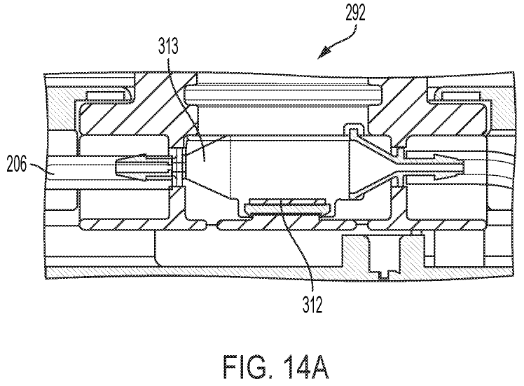

Referring to FIG. 13, an exemplary PSS flow path is depicted within the base 100 of the fetal chamber assembly 10. It will be appreciated that other suitable flow paths may be used, and the exact arrangement of the flow path as shown in the figures is not intended to be limiting. The PSS is introduced into the fetal chamber assembly 10 from a PSS source and is split into two separate inlets: a first inlet 194 and a second inlet 198. As briefly explained above, the first inlet 194 is defined in the growth chamber 120, such that the PSS from the first inlet 194 is moved into the growth chamber 120, and the second inlet 198 is defined at the cannula entrance 162 of the cannulation chamber 150, such that the PSS from the second inlet 198 is moved into the cannulation chamber 150. The PSS is configured to move generally along the longitudinal direction y towards an outlet 202. The outlet 202 is spaced from the first and second inlets 194, 198 along the longitudinal direction y. In some aspects, the outlet 202 is disposed in the growth chamber 120 opposite the first inlet 194, such that the fetus may be positioned between the first inlet 194 and the outlet 202. The first inlet 194 may be disposed in the growth chamber 120 closer to the head of the fetus than to the feet of the fetus, while the outlet 202 may be disposed such that it is closer to the feet of the fetus than the head of the fetus. This allows the PSS that flows from the first inlet 194 towards the outlet 202 to generally flow in the direction from the fetus's head towards the fetus's feet. Although the fetal chamber assembly 10 may be rotated along different axes, as will be discussed in detail below, it may be preferable to maintain orientation of the fetal chamber assembly 10 such that the outlet 202 is disposed at the lowest point of the growth chamber 120 (relative to gravity) so that the PSS may flow downward, due to gravity, towards the outlet 202. Such a flow path may be advantageous in keeping contaminants (e.g. meconium) away from the fetus's head by having a continuous flow of PSS that can move any contaminants towards the feet and towards the outlet 202, rather than move them towards or keep them adjacent to the fetus's head. Aspiration of contaminants (such as meconium) may result in respiratory complications or otherwise interfere with the fetus's development, so it may be preferable to maintain a flow of PSS that direct any contaminants or foreign bodies away from the fetus's head.

An outlet channel 206 extends from the outlet 202 and leads to a waste receptacle configured to receive the PSS after it has moved through and out of the fetal chamber assembly 10. The outlet channel 206 may be disposed, at least partly, within the housing 108. The outlet channel 206 may be configured such that the PSS flowing therethrough can contact, flow adjacent to, or flow through one or more components of the fetal chamber assembly 10. Referring to FIG. 13, for example, a meconium sensor assembly 292 may be disposed on or adjacent to the outlet channel 206, such that the PSS liquid flowing through the outlet channel 206 is subject to sensing by the meconium sensor assembly 292, as will be discussed in detail below.

In some aspects, the fetal chamber assembly 10 may include a plurality of outlets 202. Each outlet 202 may be configured to fluidly communicate with the same outlet channel 206, or, alternatively, may be configured to fluidly communicate with separate outlet channels 206.

In operation, the PSS enters the fetal chamber assembly 10 at the first and second inlets 194, 198 and flows towards the outlet 202. Although the fetal chamber assembly 10 is depicted having a dividing wall 158 that separates the growth chamber 120 and the cannulation chamber 150, it should be understood that the dividing wall 158 may have different dimensions in different embodiments, and the flow of the PSS liquid may be affected by the specific arrangement of the dividing wall 158. For example, as can be seen in FIGS. 2-4, the dividing wall 158 extends from the housing 108 upwards (towards the lid 112 when the fetal chamber assembly 10 is closed) along the vertical direction z. In some preferred aspects, the dividing wall 158 may be configured to extend in the vertical direction z such that the top of the dividing wall 158 is between the housing 108, from which the dividing wall 158 extends, and a plane, defined by the lateral and longitudinal directions x and y, in which the top surface of the seal 296 is disposed. Simply put, the height of the dividing wall 158 (measured in the vertical direction z from the housing 108) is less than the height of the seal 296. In such embodiments, when the fetal chamber assembly 10 is closed and the lid 112 is sealingly secured with the base 100, the PSS liquid may pass over the dividing wall 158 in the space defined between the dividing wall 158 and the lid 112. Such embodiments may be preferred to decrease areas of stagnant liquid within the fetal chamber assembly 10, which, in turn, decreases prevalence of bacterial growth. Additionally, such embodiments may make closing the fetal chamber assembly 10 simpler, as only a single seal 296 may be used. In some alternative aspects, the dividing wall 158 may be configured to have a height such that the top of the dividing wall 158 matches the height of the seal 296, so that when the fetal chamber assembly 10 is closed, no space is defined between the dividing wall 158 and the lid 112, and the PSS is not permitted to pass over the dividing wall 158.

The PSS may be introduced into the fetal chamber assembly 10 from a single source and using a single pump. At the fetal chamber assembly 10, the PSS may be split into two (or more) inlets as described above. In some preferred embodiments, each inlet does not have a separate pump or similar mechanism for moving the PSS therethrough independently of the other inlet. As such, the distribution of quantity of PSS between individual inlet ports does not need to be actively controlled. Referring still to FIG. 13, each of the first and second inlets 194, 198 may be configured to receive either the same amount of PSS or different amounts of PSS depending on the parameters of the fetal chamber assembly 10. Similarly, the PSS being introduced through each of the first and second inlets 194, 198 may have either substantially the same pressure or may have different pressures.

In some aspects, the quantity of the PSS that is introduced into each of the first and second inlets 194, 198 may depend on the position of the fetal chamber assembly 10, and more specifically on the position of the first and second inlets 194, 198 relative to each other. The distribution of PSS among different inlets may depend on the pressure difference of the PSS as it is directed to each inlet. The relative position of each inlet may change based on how the fetal chamber assembly 10 is disposed; the fetal chamber assembly 10 may be translated in 1, 2, or 3 directions and may be rotated along a plurality of axes. For purposes of this discussion, the fetal chamber assembly 10 may be translated along the lateral direction x, along the longitudinal direction y, and/or along the vertical direction z. The fetal chamber assembly 10 may be rotated along a pitch axis that is parallel to the lateral direction x, along a roll axis parallel to the longitudinal direction y, and/or along a yaw axis parallel to the vertical direction z. The specific location of each of the pitch, roll, and yaw axes relative to the fetal chamber assembly 10 may differ between various embodiments and is not intended to limit the description below unless indicated otherwise. The fetal chamber assembly 10 may be configured to rotate around other axes as well, and embodiments in this disclosure are not limited to the pitch, roll, and yaw axes described above.

For example, referring to the exemplary arrangement of the first and second inlets 194, 198 shown in FIG. 13 (also seen in FIG. 3), the first inlet 194 and the second inlet 198 are shown to be in the same plane defined by the lateral direction x and the longitudinal direction y. In such an arrangement, the PSS that is introduced to the two inlets may have the same pressure. As such, the flow rate of the PSS may be equal at the first inlet 194 and at the second inlet 198. If the fetal chamber assembly 10 is rotated about the roll axis in a first direction, one of the first and second inlets 194, 198 will be disposed higher (along the vertical direction z and relative to ground) than the other of the first and second inlets 194, 198. The fetal chamber assembly 10 may be rotated about the roll axis in a second direction opposite the first direction, such that the relative arrangement of the first and second inlets 194, 198 above is reversed. The inlet that is higher will have a lower pressure of PSS than the inlet that is lower. The farther the fetal chamber assembly 10 is rotated along the roll axis, the greater the relative distance becomes between the first and second inlets 194, 198, and the greater the pressure difference becomes. Whichever of the first and second inlets 194, 198 is relative lower than the other of the inlets will receive proportionally more of the PSS liquid therein compared to the other inlet. Exemplary, non-limiting pitch and roll axes according to one embodiment are depicted in FIG. 43.

This distribution may be due to the mechanism configured to introduce the PSS to the fetal chamber assembly 10 (e.g. a pump). The pump may be configured to move the PSS to the fetal chamber assembly 10 but not actively guide the flow into a specific inlet—that is, the pump is configured to move the PSS liquid into the fetal chamber assembly 10, but the liquid will flow in the direction of least resistance. When the first and second inlets 194, 198 are in the same horizontal plane defined by the lateral direction x and the longitudinal direction y, the flow may move into both inlets evenly because they both have the same resistance. When the fetal chamber assembly 10 is rotated along the roll axis in the first direction, the inlet that is higher along the vertical axis z (relative to the ground) has a greater resistance to flow than the inlet that is relatively lower, as it is harder to move the liquid higher against gravity than to a point that is relatively lower.

In some aspects, a second mechanism for moving the liquid (e.g. a second pump) may be disposed in fluid communication with the outlet channel 206 and may be configured to facilitate movement of the PSS in the outlet channel 206 out of the fetal chamber assembly 10.

### Meconium Sensing

During gestation, the fetus may sometimes release meconium into its immediate environment. While meconium itself is generally sterile, its presence in the fetal chamber assembly 10 may increase the risk of bacterial growth. Meconium may clog or damage components within the fetal chamber assembly 10 and may interfere with development of the fetus. In some instances, the fetus may aspirate the meconium, which may cause health problems for the fetus, such as infection. As such, it is desirable to monitor the fetal chamber assembly 10 during its operation for presence of meconium. If meconium is detected, it may be removed from the fetal chamber assembly 10 as will be discussed in detail below.

As shown in FIGS. 2-5, a meconium sensor assembly 292 may be disposed on the base housing 108 of the base 100. The meconium sensor assembly 292 is configured to detect presence of meconium within the liquid (e.g. PSS) that is flowing through the fetal chamber assembly 10. It will be appreciated that the fetal assembly 10 may include a plurality of strategically placed meconium sensor assemblies 292, for example, within the cannulation chamber 150, within the growth chamber 120, or in another portion of the fetal chamber assembly 10.

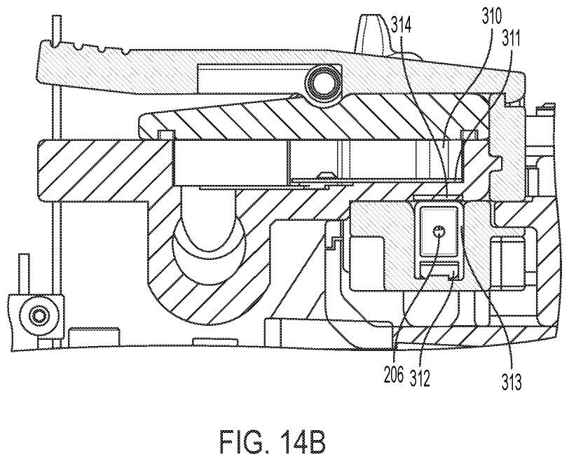

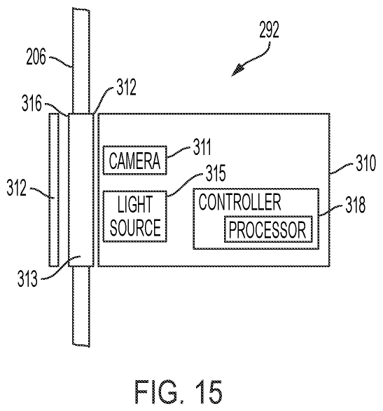

In some preferred embodiments, as shown in FIGS. 13, 14A, and 14B, for example, the meconium sensor assembly 292 may be disposed within or adjacent to the outlet channel 206. The meconium sensor assembly 292 may be in-line with the outlet channel 206. FIG. 15 depicts an exemplary, nonlimiting schematic of a sample arrangement of a meconium sensor assembly 292 as it is disposed adjacent to the outlet channel 206. It will be appreciated that this schematic is not shown to scale, and that other arrangements may be utilized. The meconium sensor assembly 292 includes a meconium sensor assembly housing 313 and a sensor 310. The liquid in the outlet channel 206 may enter the sensor assembly housing 313. The sensor 310 is configured to detect any presence of meconium within the liquid in the sensor assembly housing 313. It will be appreciated that a specific threshold amount of meconium may be predetermined for operation of the fetal chamber assembly 10. As liquid enters the outlet channel 206 at the outlet 202, the liquid travels along the outlet channel 206 and exits the fetal chamber assembly 10. After the liquid is moved into the outlet channel 206, the liquid may pass through or adjacent to the meconium sensor assembly 292.

If the sensor 310 detects presence of meconium in the liquid that surpasses the predetermined threshold, the meconium sensor assembly 292 may cause the fetal chamber assembly 10 to notify the user, trigger an alarm, or modify its operation in response to the detected meconium. Placing the meconium sensor assembly 292 within the outlet 206 may be advantageous for accurate detection of meconium due to the flow of liquid through the fetal chamber assembly 10. As explained above, the flow of liquid moves generally in the direction from the first and second inlets 194, 198 towards the outlet 202, and, as such, generally in the direction from the fetus's head towards the fetus's feet. Any meconium that the fetus excretes may be carried by the flow of liquid towards the outlet 202 and into the outlet channel 206. As explained above, the relative arrangement of the inlets 194, 198 and outlet 202, as well as the general shape of the growth chamber 120 and the cannulation chamber 150, help decrease instances of stagnant liquid and areas within the growth or cannulation chambers 120, 150 where bacteria can proliferate. As such, the advantageous design and placement of components may also help direct most or all of the excreted meconium into the outlet 202, such that the amount of meconium detected by the meconium sensor assembly 292 represents a more accurate amount of meconium that is excreted by the fetus.

As briefly noted above, the meconium sensor assembly 292 includes a sensor assembly housing 313 and a sensor 310. The sensor 310 may be a spectral sensor that includes a camera 311 configured to be pointed at a reflector surface 312. The reflector surface 312 may be a Lambertian reflector. The reflector surface 312 may include polytetrafluoroethylene (PTFE). In some aspects, the reflector surface 312 may include a single color. In some aspects, the reflector surface 312 may be white. A light source 315 may be disposed on the camera 311 or adjacent thereto at a specified distance from the reflector surface 312. The light source 315 can direct light at the reflector surface 312 such that at least a portion of the light reflects from the reflector surface 312 towards the camera 311. The camera 311 is arranged opposite the reflector surface 312, such that the sensor assembly housing 313, which includes the liquid flowing therethrough, is disposed between the camera 311 and the reflector surface 312. In some aspects, the outlet channel 206 may extend through or be in line with the sensor housing 213.

The liquid moving through the outlet channel 206 can move into the sensor assembly housing 313 adjacent the sensor 310. When in the sensor assembly housing 313, the liquid can therefore pass between the camera 311 and the reflector surface 312. The camera 311 may be a single-pixel camera configured to detect an optical change (relative to predetermined values) in the fluid between the camera 311 and the reflector surface 312. For example, in some aspects, the camera 311 may be configured to detect the relative intensity of two or more wavelengths. The camera 311 has to be able to view the reflector surface 312; as such, the materials between the camera 311 and the reflector surface 312 should be at least translucent enough for the camera to see and detect color of the reflector surface 312. This arrangement allows for the reflection of light from any material that may be present within the liquid passing thorough the outlet channel 206, as well as from the reflector surface 312, which serves as a constant backdrop to measure the spectral footprint against. In some embodiments, the sensor assembly housing 313 may include a first transparent or translucent window 314 disposed on the sensor assembly housing 313 between the camera 311 and the reflector surface 312. A second transparent or translucent window 316 may be disposed on the sensor assembly housing 313 opposite the first window 314 and also between the camera 311 and the reflector surface 312. The camera 311 may be configured to view the reflector surface 312 through the first window 314, through the sensor assembly housing 313 and the liquid therein, and through the second window 316. It will be appreciated that, in some aspects, additional windows may be arranged on the sensor 310, the sensor assembly housing 313, or elsewhere on the fetal chamber assembly housing 108.

The sensor 310 may include a controller 318 having a processor configured to use the camera 311 to detect a change in color that is different from the reflector surface 312. The processor may include a program that defines a preferred color spectrum range of interest. Different materials or components that are positioned between the camera 311 the reflector surface 312 may have different colors. In the preferred embodiments, the processor may be configured to identify a color range consistent with color of meconium. In some aspects, the color range may include red, yellow, brown, combinations of the above, or related colors. If color within the programmed range is detected, it may be indicative of presence of a particular material. In the preferred aspects, for example, if the camera 311 detects a red, yellow, brown, or similar color, this may be indicative of presence of meconium.

In some aspects, the sensor 310 may be configured to detect six different wavelengths within a visible or near-infrared spectrum. Visible spectrum has the capacity to convert individual spectrum readings to RGB or HSV values. In some aspects, HSV may have a benefit over RGB of having an intuitive method of interpreting color by using color mapping to a 3D polar space based on measured hue, saturation, and value. In such exemplary aspects, measurement of the hue may be used to quantify the detected color in a 360 degree space; measurement of saturation may be used to quantify the amount of color as a percentage; and measurement of value may be used to quantify brightness as a percentage. Regions in the 360 degree space may be associated with specific materials (e.g. meconium or blood). Such measurements allow for accurate detection and quantity of the material of interest. Configuring a processor to focus on the relevant region in the HSV space allows for monitoring of the specific materials of interest while disregarding presence of materials not of interest that may be associated with other regions of the HSV space.

In some aspects, the blood sensor may be an optical sensor that detects the presence of blood by the absorption of specific spectral lines by the blood constituents and the relative intensity of specific wavelengths. The sensor may emit different wavelengths alternately and detect the transmitted or reflected intensity. The sensor may emit multiple wavelengths simultaneously and filtered detectors measure the intensity of specific wavelengths.

If the camera 311 detects presence of a color within the programmed color range, the controller 318 may be configured to notify the user, trigger an alarm, or modify operation of the fetal chamber assembly 10. It will be appreciated that the liquid flowing through the outlet channel 206 may include various colors, and so configuring the sensor to focus only on colors pertaining to the material being monitored (e.g. meconium or blood) can help prevent false-positives.

### Meconium Removal

Meconium that is excreted by the fetus into the growth chamber 120 may be removed from the fetal chamber assembly 10 to reduce risk of infection, bacterial growth, or damage of assembly components. The amount of meconium inside the fetal chamber assembly 10 may be estimated by the meconium sensor assembly 292 as described above. Meconium may be visible within the growth chamber 120 and/or the cannulation chamber 150. In some aspects, it may be advantageous to remove the meconium if the amount of meconium detected by the sensor 310 in the meconium sensor assembly 292 exceeds a predetermined threshold.

While it is possible to open the fetal chamber system 10 (e.g. by de-coupling the lid 112 from the base 100) and removing the meconium from within the liquid (e.g. PSS) therein, it may be preferable to remove the meconium without unlocking and opening the fetal chamber system 10. This helps maintain the controlled environment for the fetus without disturbing the fetus, exposing the fetus or the interior of the fetal chamber assembly 10 to external contaminants, or pausing operation of the fetal chamber assembly 10 (e.g. without pausing continuous flow of the liquid through the assembly). As such, in some aspects, it may be preferable to remove meconium via a dedicated removal port through which a user may insert a tool into the growth chamber 120 or cannulation chamber 150 and suction, scoop, or otherwise remove meconium present in the liquid. As shown in FIGS. 2-5, a meconium removal assembly 214 may be disposed on the base 100.

Although the above description provides examples of removing meconium specifically from the growth chamber 120, it will be understood that meconium may be present in other parts of the fetal chamber assembly 10, such as the cannulation chamber 150, and it may be removed via the disclosed meconium removal assembly 214 from those regions as well. In some aspects, the fetal chamber assembly 10 may include additional meconium removal assemblies 214 disposed advantageously on the fetal chamber assembly 10 to allow access to regions where meconium may be present.

The meconium sensor assembly 292 can be configured to detect color changes indicative of presence of meconium, as described above. In some aspects, the meconium sensor assembly 292 may additionally be configured to detect changes of color corresponding to presence of blood in the liquid passing through the outlet channel 206. The presence of blood in the liquid that exits the growth chamber 120 may be indicative of fetal bleeding. Blood in the growth chamber 120 or the cannulation chamber 150 may indicate a leak between one or more cannulated vessels in the umbilical cord and the respective cannulas connected thereto. It is preferable to monitor the fetal chamber assembly 10 for presence of blood and to address such a problem before the fetus can be injured.

### Temperature Sensing

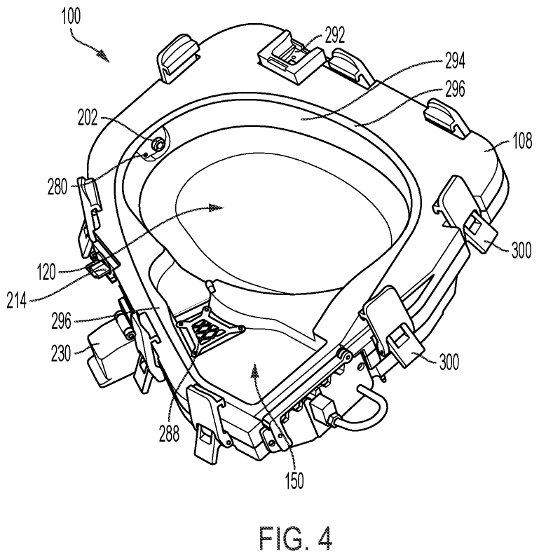

In addition to blood and meconium, the fetal chamber assembly 10 may be configured to monitor various other parameters of the liquid (e.g. PS S) flowing therethrough. In some aspects, one or more temperature sensors may be disposed throughout the fetal chamber assembly 10 to measure temperature of the liquid, components of the fetal chamber assembly 10, or the fetus itself. In some aspects, the fetal chamber assembly 10 may include a plurality of temperature sensors arranged strategically throughout the fetal chamber assembly 10 to provide an accurate measurement of temperature. As shown in FIGS. 3 and 4, the fetal chamber assembly 10 may include temperature sensors 280 disposed within growth chamber 120 to measure the liquid therein. The temperature measurements from some or all of the plurality of temperature sensors may be analyzed to calculate an average temperature within the fetal chamber assembly 10, to determine temperature differences in various areas of the fetal chamber assembly 10, to confirm temperature sensor function, and/or to monitor specific regions individually.

Referring to FIG. 42, an exemplary layout of three temperature sensors 280 is depicted. Although FIG. 42 shows three temperature sensors 280, it should be understood that the fetal chamber assembly 10 may be designed with a different number of temperature sensors. For example, 1, 2, . . . 10, or another suitable number of temperature sensors 280 may be envisioned. Additionally, “secondary” temperature sensors 280 may be arranged as redundancies in the event one or more “primary” temperature sensors 280 become un-operational or defective. Primary and secondary temperature sensors may be substantially the same, with the difference being in intended use.

The temperature sensors 280 may be disposed partly or entirely inside the growth chamber 120, the cannulation chamber 150, and/or a fluid channel within the housing 108. The specific arrangement will depend on which region the particular temperature sensor 280 is intended to monitor. As shown in FIG. 42, in some embodiments, the fetal chamber assembly 10 may include three temperature sensors 280 disposed in various regions of the growth chamber 120. For purposes of this disclosure, the three temperature sensors 280 in FIG. 42 are individually labeled as a first temperature sensor 280a, a second temperature sensor 280b, and a third temperature sensor 280c. It will be appreciated that the first, second, and third temperature sensors 280a-c may be functionally and structurally the same. The first temperature sensor 280 may be arranged adjacent the first inlet 194. When the fetus is disposed in the growth chamber 120, the first temperature sensor 280 will be the closest of the three depicted temperature sensors to the fetus's head. It may be advantageous to have an accurate measurement of the temperature of liquid in the region of the fetus's head. Additionally, the placement of the first temperature sensor 280 adjacent to the first inlet 194 can allow for accurate sensing of temperature of the liquid as the liquid first enters the growth chamber 120.

The second temperature sensor 280b may be disposed adjacent the opening 166 between the cannulation chamber 150 and the growth chamber 120. The second temperature sensor 280b may be adjacent to the meconium removal port 218. The second temperature sensor 280b may be disposed at least partially within the growth chamber 120 between the opening 166 and the meconium removal port 218. Such placement may be advantageous as it allows for accurate temperature monitoring immediately downstream of where the liquid from the cannulation chamber 150 enters the growth chamber 120 and mixes with the liquid within the growth chamber 120. Monitoring the temperature in this region allows for making sure liquid that enters the cannulation chamber through the second inlet 198 is sufficient temperature. In some aspects, it may be advantageous to monitor temperature adjacent the meconium removal port 218. If, during operation, meconium is removed via the meconium removal port 218, as described in detail above, it may be advantageous to monitor the liquid in the immediate vicinity of the meconium removal port 218 to detect any change in temperature caused by the opening of the port.

The third temperature sensor 280c may be disposed adjacent to the outlet 202. The third temperature sensor 280c may be arranged opposite the first temperature sensor 280a and may be separated from the first temperature sensor 280a along the longitudinal direction y. The third temperature sensor 280c may be arranged such that the second temperature sensor 280b is disposed between the first and third temperature sensors 280a, 280c. When the fetus is disposed in the growth chamber 120, the third temperature sensor 280c may be the closest of the three temperature sensors to the fetus's feet. It may be advantageous to measure temperature in the region of the fetus's feet and to compare the measurement with the temperature at the fetus's head measured by the first temperature sensor 280a. This may indicate how the temperature of the liquid flowing in the direction from the fetus's head towards the fetus's feet changes. Placing the third temperature sensor 280c adjacent the outlet 202 may be advantageous to measure temperature of the liquid as it exits the growth chamber 120 and to compare the measurement to the temperature of the liquid as it enters the growth chamber at the first inlet 194 and/or at the opening 166. It will be appreciated that the specific exemplary arrangement of the three temperature sensors 280a-c is not intended to be limiting, and that other arrangements of, as well as greater or fewer quantities of, temperature sensors 280 are envisioned. In some aspects, a temperature sensor 280 may be disposed in the cannulation chamber 150, for example adjacent to the second inlet 198.

In operation, it is preferable to maintain the temperature of the liquid inside the fetal chamber assembly 10 within a preferred temperature range. It will be appreciated that temperature of the fetal environment can affect growth and development of the fetus, and that temperatures outside of a preferred range can cause injury to the fetus. As such, in some embodiments, it is preferable to maintain the temperature of the liquid in the growth chamber 120 and in the cannulation chamber 150 to be approximately 37.5 degrees Celsius (C). Variations of temperature may be permissible, and the exact preferred temperature may be varied depending on medical requirements pertaining to the fetus.

The fetal chamber assembly 10 may be configured to cause the entering liquid to be heated or cooled to a desired temperature based on the temperature measurements from the one or more temperature sensors 280. For example, if an individual measurement or an average measurement of temperature is lower than a predetermined threshold, the fetal chamber assembly 10 may be configured to cause the liquid to be heated sufficiently to raise the temperature of the liquid to the desired temperature; conversely, if an individual or average measurement of temperatures is higher than a predetermined threshold, the fetal chamber assembly 10 may be configured to cause the liquid to be cooled sufficiently (or, alternatively, to not be heated) such that the temperature of the liquid is lowered to the desired temperature.

In some aspects, additional temperature sensors (not shown on the fetal chamber assembly 10) may be disposed outside of the fetal chamber assembly 10 to measure temperature of the liquid moving into the fetal chamber assembly 10. These additional temperature sensors may be used to monitor the temperature of the liquid to make sure the liquid is heated or cooled to a desired temperature before it is introduced into the fetal chamber assembly 10.

### Pressure Sensing

The fetal chamber assembly 10 may be configured to monitor pressure therein. One or more pressure sensors may be disposed throughout the fetal chamber assembly 10 to measure pressure of the liquid in the growth chamber 120, the cannulation chamber 150, at the first inlet 194, at the second inlet 198, at the outlet 202, at the outlet channel 206, or at another region of the fetal chamber assembly 10. In some aspects, the fetal chamber assembly 10 may include a plurality of pressure sensors arranged strategically throughout the fetal chamber assembly 10 to provide an accurate measurement of pressure. The fetal chamber assembly 10 may be configured to utilize measurements from each of the plurality of pressure sensors to determine an average pressure calculation. As shown in FIG. 13, the fetal chamber assembly 10 may include pressure sensors 284 disposed therein.

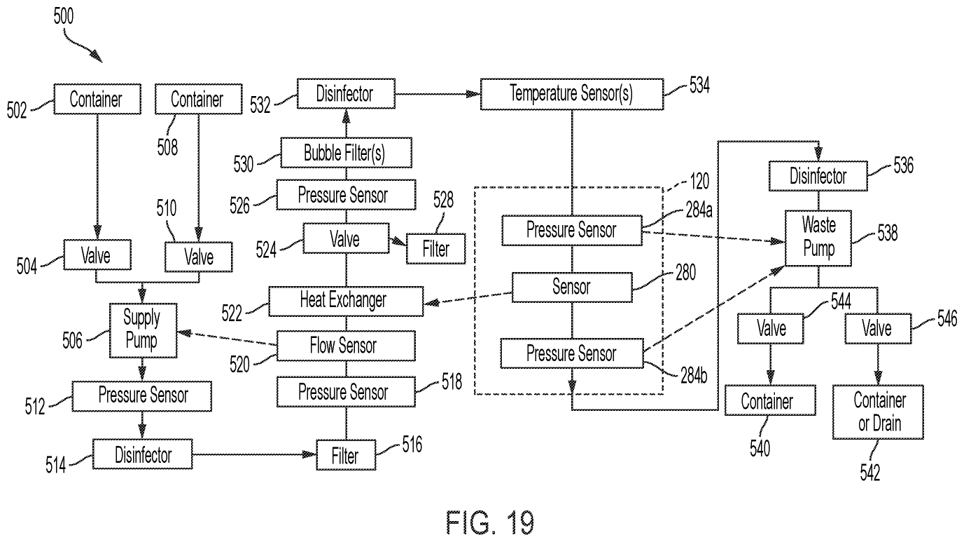

Referring to FIG. 19, an exemplary layout of two pressure sensors 284 is depicted. Although FIG. 19 shows two pressure sensors 284, it should be understood that the fetal chamber assembly 10 may be designed with a different number of pressure sensors (e.g., 284a, 284b, etc.). For example, 1, 2, . . . 10, or another suitable number of pressure sensors 284 may be envisioned. Additionally, “secondary” pressure sensors 284 may be arranged as redundancies in the event one or more “primary” pressure sensors 284 become un-operational or defective. Primary and secondary pressure sensors may be substantially the same, with the difference being in intended use.

Referring to FIG. 13, the fetal chamber assembly 10 may be configured to receive measurements from each pressure sensor 284 and to make a calculation based on each individual measurement. The separate measurements may be used to calculate an average pressure within a component of the fetal chamber assembly 10 or a pressure at a particular position relative to the sensors. In some aspects, the values at each pressure sensor 284 can be used to calculate the pressure at the geometric mid-point of the growth chamber 120 or at another preferred region in the growth chamber 120. In some scenarios, it is preferable to continuously monitor the average pressure within the growth chamber 120, specifically when the fetus is disposed therein. As shown in FIG. 13, in some embodiments, the fetal chamber assembly 10 may include two pressure sensors 284 disposed around the growth chamber 120 according to a preferred arrangement. For purposes of this disclosure, the two pressure sensors 284 shown in FIG. 13 are individually labeled as a first pressure sensor 284a and a second pressure sensor 284b. It will be appreciated that the first and second pressure sensors 284a, 284b may be functionally and structurally the same. The use of multiple pressure sensors 284 can advantageously provide pressure readings of a specific area or zone within the growth chamber 120, and the specific regions being monitors may depend on the position of the fetus within the growth chamber 120 relative to the separate pressure sensors 284. Pressure within the fetal chamber assembly 10 can be regulated in response to monitored pressure based on individual pressure measurements at one or more of the plurality of pressure sensors 284 and/or based on a calculated pressure value that is calculated based on the pressure measurements from one or more individual pressure sensors 284.

In some embodiments, the first and second pressure sensors 284a, 284b may be disposed such that each is essentially equidistant from the physical centroid of the growth chamber 120. In some embodiments, the first and second pressure sensors 284a, 284b may be disposed such that each is essentially equidistant from the pitch axis A. Specifically, the first pressure sensor 284a may be disposed adjacent to the portion of the growth chamber 120 that will receive the head of the fetus, and the second pressure sensor 284b may be disposed adjacent to the portion of the growth chamber 120 that will receive the feet of the fetus. That is, the first pressure sensor 284a may be closer to the head of the fetus than to the feet of the fetus. The second pressure sensor 284b may be closer to the feet of the fetus than to the head of the fetus.

The fetal chamber assembly 10 may be configured to notify a user, trigger an alarm, and/or modify position or operation thereon if the measured pressure is outside of a predetermined range. In some aspects, it may be preferable to maintain the pressure within the growth chamber 120 (calculated at the centroid of the growth chamber) between from about 4 mmHg to about 6 mmHg. It will be appreciated that other suitable pressure ranges may be utilized and will depend on parameters of the fetal chamber assembly 10 and the fetus.

Pressure Relief

In some aspects, gas may become trapped within the fetal chamber assembly 10 during loading of the fetus, during the cannulation process, during removal of meconium, or during movement of the fetal chamber assembly 10. The gas may include air and may include a common mixture of atmospheric gases. In some aspects, air may seep inside the fetal chamber assembly 10 at one or more of the ports described throughout this application. Furthermore, dissolved gases in the liquid being moved into and through the fetal chamber assembly 10 may separate out of the liquid. During operation of the fetal chamber assembly 10, gases may escape from the fetus during normal gestation processes and enter the environment immediately adjacent the fetus (i.e. the liquid surrounding the fetus in the growth chamber 120). The air (or other gases) may be disposed, in gaseous form, between the base 100 and the lid 112. In some aspects, pockets of air may form in the growth chamber 120 and/or in the cannulation chamber 150.

Air present within the growth chamber 120 and/or the cannulation chamber 150 may be hazardous to the fetus. In some aspects, presence of air may interfere with desired imaging of the fetus during gestation. For example, air may impede ultrasound imaging of the fetus in the growth chamber 120. In some aspects, presence of air may lead to drying of assembly components, tubing, cannulas, and the like. This may lead to physical cracks or breaks in the components, which may cause leaks within the fetal chamber assembly 10. It is preferable to keep the fetus and its umbilical cord submerged within liquid during the gestation process. If parts of the fetus or its umbilical cord contact the air, the fetus or the umbilical cord may dry out or otherwise damaged. Further, the air trapped inside the fetal chamber assembly 10 may be non-sterile and may contain contaminants, viruses, bacteria, or other impurities that are undesirable within the fetal chamber assembly 10.

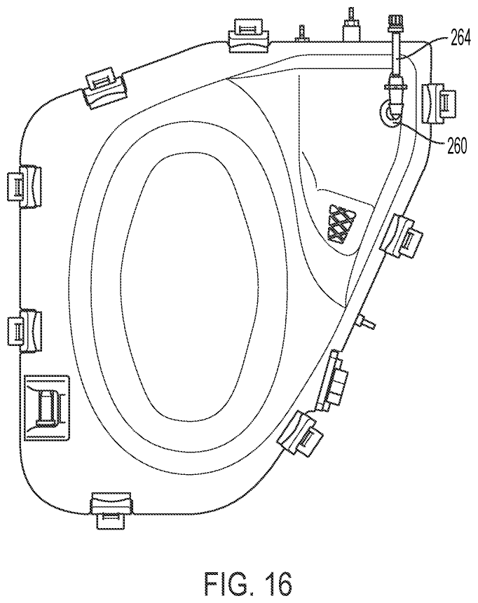

It may be preferable to remove at least a portion of the air trapped within the fetal chamber assembly 10. The fetal chamber assembly 10 can include a pressure relief element configured to reduce pressure within the growth chamber 120. Referring to FIG. 16, the air may be moved out of the growth chamber 120 and/or the cannulation chamber 150 through one or more air removal ports 260 disposed on the fetal chamber assembly 10 (see, generally, FIG. 2 and FIG. 16). Referring to FIG. 2, an air removal port 260 may be disposed on the base 100 or on the lid 112. The air removal port 260 can be the pressure relief element. In some embodiments, the air removal port 260 may be disposed on the cannulation chamber membrane 308 (as shown in FIG. 2). In some embodiments, the air removal port 260 may be disposed on the top membrane 124 of the growth chamber 120. In some further embodiments, the air removal port 260 may be disposed on the housing 108 of the base 100 (see, also, FIG. 2). In some aspects, the fetal chamber assembly 10 may include a plurality of air removal ports 260 disposed throughout the fetal chamber assembly 10. In certain embodiments, the air port 260 can be used to remove a fluid (e.g., gas or liquid such as PSS).

Each air port 260 defines a passage extending therethrough that fluidly communicates between the interior space 104 of the fetal chamber assembly 10 (i.e. the space between the base 100 and lid 112) or the growth chamber and the environment outside of the fetal chamber assembly 10. Because the liquid that will be flowed through the fetal chamber assembly 10 is heavier and denser than air, the liquid (e.g. PSS) will naturally fall downward (with gravity) and displace air, such that air is located relatively above the liquid (“above” being measured from the liquid in the direction against gravity). Due to the shapes of components of the fetal chamber assembly 10, air bubbles that are formed may be trapped in a region of the fetal chamber assembly 10 that does not include an air removal port 260. As such, it may be preferable to move the fetal chamber assembly 10 such that the trapped air bubbles are directed towards the one or more air removal ports 260. As explained previously, the fetal chamber assembly 10 may be rotated along the pitch, roll, and yaw axes. In operation, a user can rotate the fetal chamber assembly 10 along one, two, or all three of the pitch, roll, and yaw axes to direct the trapped air bubbles to the desired air removal port 260. In some exemplary embodiments the fetal chamber assembly 10 may be rotated along the roll axis up to approximately 45 degrees (measured from the transverse-longitudinal plane defined earlier) such that air trapped between the base 100 and the lid 112 is moved towards the air removal port 260 disposed on the cannulation chamber membrane 308. As the air is moved adjacent the air removal port 260, the air may flow through the air removal port 260 and out of the fetal chamber assembly 10.

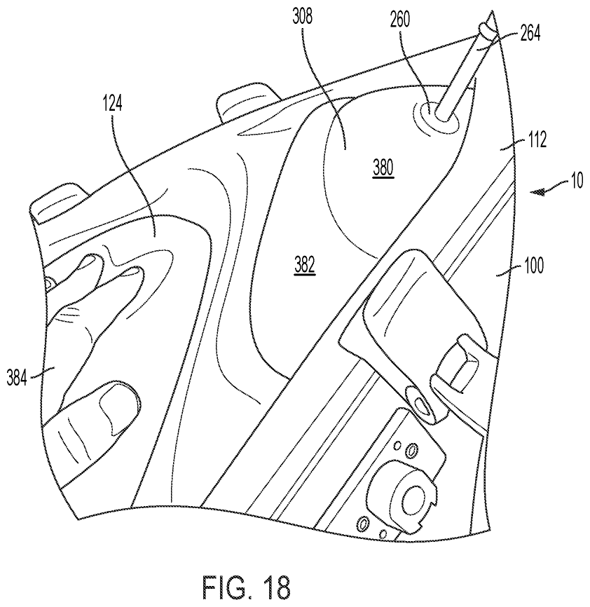

In some aspects, the user may deform, push, or palpate the top membrane 124 or the cannulation chamber membrane 308 to direct the air in the desired direction towards the air removal port 260. In some aspects, an air removal port 260 may be disposed on the housing 108. For example, the air removal port 260 may be disposed adjacent to the meconium removal port 218. Referring to FIG. 18, an exemplary arrangement of a fetal chamber assembly 10 is depicted. The fetal chamber assembly 10 is shown having been rotated along the roll axis to a desired angle. An air bubble 380 can be seen disposed adjacent to the air removal port 260. Liquid 382 is shown beneath the air bubble 380 (“beneath” being relative to the vertical direction in the direction of gravity). A user 384 is shown applying force to the top membrane 124. The force and the relative position of the fetal chamber assembly 10 causes the air bubble 380 to be moved towards the air removal port 260, where the air may be discharged from the fetal chamber assembly 10.

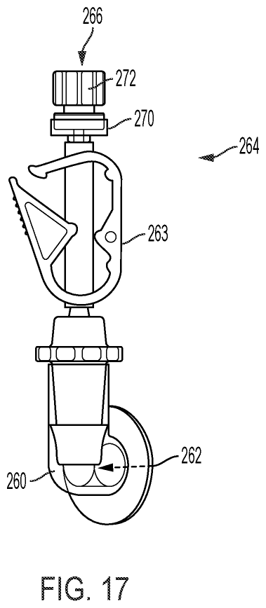

The air removal port 260 may be configured to receive an air removal assembly 264 therein. The air removal assembly 264 allows for selectively opening and closing the air removal port 260, such that air may pass through or be precluded from passing through, respectively. Referring to FIGS. 16-17, an exemplary air removal assembly 264 is depicted engaged with an exemplary air removal port 260. It will be appreciated that other similar devices may be utilized. The air removal port 260 includes a passage 262 extending therethrough that fluidly communicates with both the interior surface 104 and the environment outside of the fetal chamber assembly 10. The air removal port 260 is configured to receive an air removal assembly 264 into the passage 262. The air removal assembly 264 defines a passage 266 extending therethrough. The passage 266 is configured to be in fluid communication with the passage 262. When the air removal assembly 264 is engaged with the air removal port 260, the passage 266 is in fluid communication with the interior space 104 and the environment outside of the fetal chamber assembly 10. The air removal assembly 264 may include a clamp 268 configured to selectively block or unblock the passage 266. It will be understood that the material of the air removal assembly 264 should be deformable enough such that it may be compressed by the clamp 268 and resilient enough to return to an uncompressed position when the clamp 268 is opened. The air removal assembly 264 may comprise a plastic or silicone tube. The air removal assembly 264 may further include a check valve 270 configured to allow air or liquid to pass therethrough in one direction (e.g. in the direction out of the fetal chamber assembly 10) while precluding passage of materials in an opposite direction (e.g. into the fetal chamber assembly 10). A vented cap 272 may be disposed on the air removal apparatus 264 to allow air to escape from the air removal apparatus 264 through the passage 266 while preventing entrance of external contaminants or debris into the passage 266. The cap 272 may be removably connected to the air removal assembly 264 such that the cap 272 can be selectively opened or closed by the user to allow air to be removed. In some aspects, the cap 272 may be threadably connected to the air removal apparatus 264. In some aspects, the cap 272 may contain a hydrophobic filter to allow gas but not liquid to escape.

The disclosed systems and devices may be configured for use with fetuses, including term and preterm fetuses. The preterm fetus may be a premature fetus (for example, less than 37 weeks estimated gestational age, particularly 28 to 32 weeks estimated gestational age), extreme premature fetuses (24 to 28 weeks estimated gestational age), or pre-viable fetuses (20 to 24 weeks estimated gestational age). The gestation periods are provided for humans, though corresponding preterm fetuses of other animals may be used. In some aspects, the preterm fetus may have no underlying congenital disease. In other aspects, the fetus may have limited capacity for pulmonary gas exchange, for example, due to pulmonary hypoplasia or a congenital anomaly affecting lung development, such as congenital diaphragmatic hernia. The disclosed systems may be configured such that the fetus may be maintained within the system for as long as needed (for example, for days, weeks or months) until the fetus is capable of life without the system. The particular size, shape, and dimensions of the disclosed fetal chamber assemblies 10 will depend on the intended use, the size of the fetus, and manufacturing constraints. In some exemplary embodiments, the fetal chamber assembly 10 may have a first dimension measured along the longitudinal direction y of between about 10 inches and about 24 inches; between about 14 inches and about 20 inches; or in another suitable range. The fetal chamber assembly 10 may have a second dimension measured along the transverse direction x of between about 8 inches and about 22 inches; between about 12 inches and about 18 inches; or in another suitable range. The fetal chamber assembly 10 may have a third dimension measured along the vertical y direction of between about 2 inches and about 12 inches; between about 4 inches and about 10 inches; or in another suitable range.

### PSS Circuit

Referring to FIG. 19, the PSS can flow through a PSS circuit 500. The PSS circuit 500 can be configured to introduce the PSS into the growth chamber 120. The PSS circuit 500 can include a conduit that fluidly connects elements of the PSS circuit 500 to each other. The PSS circuit 500 can include a container 502. In some examples, the container 502 contains the PSS. In some other examples, the container 502 contains a substance and an aqueous solvent is passed through the container to create the PSS as previously described. The container 502 can be fluidly coupled to a valve 504. In some examples, the valve 504 is manually operable. In other examples, the valve 504 is coupled to a controller. The controller can be configured to send a signal to the valve to open or close the valve. The valve 504 can be configured to allow the PSS to flow through the valve 504 when the valve 504 is open. The valve 504 can prevent PSS flow when the valve 504 is closed. The valve 504 can be fluidly coupled to a pump 506. The pump 506 can be a supply pump. The supply pump 506 can be configured to move the PSS from the container 502 to the growth chamber 120. The supply pump 506 can be configured to regulate the PSS flow into the growth chamber 120. The PSS circuit 500 can include an integrated heat exchanger configured to regulate the temperature of the PSS within the container 502. The heat exchanger can be configured to heat the PSS within the container 502.

The container 502 can be a first container. The PSS circuit 500 can include a second container 508. The PSS circuit 500 can include a conduit that fluidly couples the containers 502, 508 to the growth chamber 120. In some examples, the second container 508 contains the PSS. In other examples, the second container 508 contains a substance and an aqueous solvent is passed through the container to create the PSS as previously described. The second container 508 can be fluidly coupled to a second valve 510. In some examples, the second valve 510 is manually operable. In other examples, the second valve 510 is coupled to the controller. The second valve 510 can be configured to allow the PSS to flow through the second valve 510 when the second valve 510 is open. The second valve 510 can prevent PSS flow when the second valve 510 is closed. The second valve 510 can be fluidly coupled to the pump 506. The first and second valves 504, 510 can be independently operable so as to allow one of the first and second containers 502, 508 to be replaced without interrupting operation of the PSS circuit 500. In some examples, the first and second containers 502, 508 include the same material (e.g. the PSS). In other examples, the first and second containers 502, 508 contain different materials that are combined by the PSS circuit 500. The PSS circuit 500 can include an integrated heat exchanger configured to regulate the temperature of the PSS within the container 508. The heat exchanger can be configured to heat the PSS within the container 508.

The PSS circuit 500 can be configured to detect a volume of the material within the first and second containers 502, 508. The PSS circuit 500 can include a sensor (e.g., weight sensor, optical sensor) configured to sense the volume of material within the first and second containers 502, 508. The PSS circuit 500 can be configured to draw from one of the first and second containers 502, 508 until a selected threshold is reached. The threshold can be a minimum weight or minimum volume. The PSS circuit 500 can be configured to close one of the first and second valves 504, 510 and open the other of the first and second valves 504, 510 when the volume of the material within the first or second container 502, 508 is at or below the selected threshold. In some examples, the controller sends first and second signals to open or close the first and second valves 504, 510. In other examples, the PSS circuit 500 generates an observable signal (e.g., sound or light) to notify a user or health care professional that one of the first and second containers 502, 508 is at or below the selected threshold.

The pump 506 can be coupled to a sensor 512. In one particular embodiment, pump 506 is a peristaltic pump. The sensor 512 can be a pressure sensor. The sensor 512 can be configured to detect pressure within the PSS circuit 500. The sensor 512 can be configured to send a sensor signal to the controller indicative of the pressure level. The controller can be configured to compare the sensor signal to a threshold level. The controller can be configured to deactivate the pump 506 if the pressure level exceeds the threshold level.

The PSS circuit 500 can include a disinfector 514 configured to disinfect the PSS as the PSS flows to toward the growth chamber 120. In some examples, the disinfector 514 includes an ultraviolet (UV) light source configured to disinfect the PSS. The conduit can be elongated along a conduit central axis. The disinfector 514 can be disposed at an angle relative to the conduit central axis so as to prevent light from traveling through the conduit to the growth chamber 120. The disinfector 514 can emit light at an angle relative to the conduit central axis of about 60 degrees to about 120 degrees, about 70 degrees to about 110 degrees, about 80 degrees to about 100 degrees, or about 90 degrees. The light source can be a light emitting diode (LED). The disinfector 514 can include a plurality of UV LEDs. The disinfector 514 can be configured to emit light having a wavelength of about 260 to about 280 nanometers. In some examples, the disinfector 514 includes a radio frequency emitter configured to disinfect the PSS. The disinfector 514 can emit radio frequency waves so as to heat the PSS, thereby disinfecting the PSS. The disinfector 514 can be configured to disinfect the PSS without contacting the PSS.

The PSS can flow through a filter 516. The filter 516 can be configured to remove particles from the PSS. The filter 516 can be configured to remove particles above a threshold size. The filter 516 can be detachably coupled to the PSS circuit. The filter 516 can be removable to replace the filter 516. The sensor 512 can be upstream from the filter 516. The sensor 512 can detect a pressure increase which can be indicative of particulate build up on the filter 516. In one embodiment, the PSS system described herein employs a plurality of filters to allow replacement of any one or more filter(s) without disturbing other filter(s) within the system.

The sensor 512 can be a first sensor. The PSS circuit 500 can include a second sensor 518. The second sensor 518 can be a pressure sensor. The first sensor 512 can be upstream from the filter 516. The second sensor 518 can be downstream from the filter 516. The second sensor 518 can be configured to detect pressure within the PSS circuit 500. The second sensor 518 can be configured to send a sensor signal to the controller indicative of the pressure level. The controller can be configured to compare the signal from the first sensor 512 to the signal from the second sensor 518. The controller can determine if the filter 516 is clogged by comparing the signals from the first and second sensors 512, 518. The controller can be configured to compare the sensor signal from the second sensor 518 to a threshold level. The controller can be configured to deactivate the pump 506 if the pressure level exceeds the threshold level.

The PSS circuit 500 can include a third sensor 520. The third sensor 520 can be a flow sensor. The third sensor 520 can be configured to send a signal that causes an increase or decrease in PSS flow from the pump 506. The third sensor 520 can be configured to send a signal directly to the pump 506. In other examples, the third sensor 520 is configured to send a signal to the controller which then sends a signal to the pump 506 in response to receiving the signal from the third sensor 520.

The PSS circuit 500 can include a heat exchanger 522. The heat exchanger 522 can be configured to heat the PSS as it flows within the PSS circuit 500. Alternatively, the heat exchanger 522 can be configured to cool the PSS as it flows within the PSS circuit 500. The heat exchanger 522 can be configured to receive a signal from the one or more temperature sensors 280 to adjust the temperature of the PSS as needed. The one or more temperature sensors 280 can be configured to send a signal to the controller. The controller can be configured to send a signal to the heat exchanger 522 in response to receiving the signal from the one or more temperature sensors 280. The heat exchanger 522 can be activated in response to the temperature sensed by the one or more temperature sensors 280. The heat exchanger 522 can include a heated mass of liquid and the PSS flows within the conduit through the heated mass of liquid so as to heat the PSS.

The PSS circuit 500 can include a third valve 524. The third valve 524 can be a diverter valve. The third valve 524 can divert the PSS flow from a first path to a second path. The first path can allow the PSS to flow from the heat exchanger 522 to a fourth sensor 526. The second path can allow the PSS to flow from the heat exchanger 522 through a second filter 528 to waste or atmosphere to relief pressure in system. In some examples, the PSS flows through each of the first filter 516 and the second filter 528 during normal operation of the PSS circuit 500. In other examples, the PSS flows through only one of the first and second filters 516, 528 during normal operation of the PSS circuit 500. A system with two filters can ensure that the PSS is filtered at all times, even when one of the first and second filters 516, 528 are removed for cleaning or replacement.

The fourth sensor 526 can be a pressure sensor. The second sensor 518 can be upstream from the second filter 528. The fourth sensor 526 can be downstream from the second filter 528. The fourth sensor 526 can be configured to detect pressure within the PSS circuit 500. The fourth sensor 526 can be configured to send a sensor signal to the controller indicative of the pressure level. The controller can be configured to compare the sensor signal to a threshold level. The controller can be configured to deactivate or otherwise adjust the PSS flow rate from the pump 506 if the pressure level exceeds the threshold level. The fourth sensor 526 can be configured to send a signal to the controller indicative of the pressure level at the fourth sensor 526. The controller can be configured to compare the signal from the second sensor 518 to the signal from the fourth sensor 526. The controller can determine if the second filter 528 is clogged by comparing the signals from the second and fourth sensors 518, 526.

The PSS circuit 500 can include a third filter 530. In one particular embodiment, the third filter 530 is a bubble filter. The third filter 530 can be downstream from each of the first and second filters 512, 528. In one particular embodiment, at least one of the first and second filters 512, 528 can be a media filter and the third filter 530 can be a bubble filter.

The PSS system in FIG. 19 contains a plurality of disinfectors. In certain embodiments of the system, any one or more of the disinfector(s) are comprise of UV light, UV light emitting diodes (“LED”), or radio frequency disinfectors. The disinfector 514 can be a first disinfector. The PSS circuit 500 can include a second disinfector 532. The second disinfector 532 can be fluidly coupled to the third filter 530. The second disinfector 532 can be configured to disinfect the PSS as the PSS flows toward the growth chamber 120. In some examples, the second disinfector 532 includes a UV light source configured to disinfect the PSS. The light source can be an LED. The second disinfector 532 can include a plurality of UV LEDs. The second disinfector 532 can be configured to emit light having a wavelength of about 260 to about 280 nanometers. The second disinfector 532 can be disposed at an angle relative to the conduit central axis so as to prevent light from traveling through the conduit to the growth chamber 120. The second disinfector 532 can emit light at an angle relative to the conduit central axis of about 60 degrees to about 120 degrees, about 70 degrees to about 110 degrees, about 80 degrees to about 100 degrees, or about 90 degrees. In some examples, the second disinfector 532 includes a radio frequency emitter configured to disinfect the PSS. The second disinfector 532 can emit radio frequency waves so as to heat the PSS, thereby disinfecting the PSS. The second disinfector 532 can be configured to disinfect the PSS without contacting the PSS. In one embodiment, the PSS system described herein employs a plurality of disinfectors to allow replacement of any one or more disinfector(s) without disturbing other disinfectors(s) within the system.

The PSS circuit 500 can include a fifth sensor 534. The fifth sensor 534 can be a temperature sensor. The fifth sensor 534 can be fluidly coupled to the disinfector 532. The fifth sensor 534 can be a plurality of sensors that measure temperature along the PSS circuit. The fifth sensor 534 can measure the temperature of the PSS as it enters the growth chamber 120. In one embodiment, the temperature of the PSS within growth chamber 120 ranges from about 37 to about 38° C.

The PSS can flow from the fifth sensor 534 to the growth chamber 120. The PSS can flow from the growth chamber 120 to a third disinfector 536. The third disinfector 536 can be fluidly coupled to the growth chamber 120. The third disinfector 536 can be configured to disinfect the PSS as the PSS flows from the growth chamber 120. Disinfecting the PSS after it exits the growth chamber 120 can allow the PSS to be disposed down a drain. The drain can be a municipal drain. In some examples, the third disinfector 536 includes a UV light source configured to disinfect the PSS. The light source can be an LED. The third disinfector 536 can include a plurality of UV LEDs. The third disinfector 536 can be configured to emit light having a wavelength of about 260 to about 280 nanometers. The third disinfector 536 can be disposed at an angle relative to the conduit central axis so as to prevent light from traveling through the conduit to the growth chamber 120. The third disinfector 536 can emit light at an angle relative to the conduit central axis of about 60 degrees to about 120 degrees, about 70 degrees to about 110 degrees, about 80 degrees to about 100 degrees, or about 90 degrees. In some examples, the third disinfector 536 includes a radio frequency emitter configured to disinfect the PSS. The third disinfector 536 can emit radio frequency waves so as to heat the PSS, thereby disinfecting the PSS. The third disinfector 536 can be configured to disinfect the PSS without contacting the PSS. The third disinfector 536 can disinfect the PSS after it exits the growth chamber 120 such that the PSS can be cycled through the PSS circuit 500 again. The third disinfector 536 can disinfect the PSS after it exits the growth chamber 120 such that the PSS can be cycled through the PSS circuit 500 again with or without adding additional PSS.

The PSS circuit 500 can include a second pump 538. The second pump 538 can be fluidly coupled to the third disinfector 536. The second pump 538 can be a waste pump that pumps the PSS to a waste container or drain. The second pump 538 can be configured to pump the PSS out of the growth chamber 120. The pressure sensors 284a, 284b of the fetal chamber assembly 10 can be configured to send a signal that control the second pump 538. In some examples, the pressure sensors 284a, 284b send a signal directly to the second pump 538. In other examples, the pressure sensors 284a, 284b send a signal to the controller that sends a signal to the second pump 538 in response to receiving the signal from the pressure sensors 284a, 284b. The second pump 538 can at least partially regulate pressure within the growth chamber 120 by adjusting or stopping the flow of PSS out of the growth chamber 120. In one particular embodiment, pressure sensors 284a and 284b maintain the pressure within growth chamber 120 at a range from about 4 to about 6 mmHg. The second pump 538 can be a first pressure reliever configured to reduce pressure within the growth chamber 120. The pressure relief element described below can be a second pressure reliever configured to reduce pressure within the growth chamber 120. The first pressure reliever can be operable independently of the second pressure reliever. The height of the PSS outlet of the growth chamber 120 can be selected to at least partially control pressure within the growth chamber 120.

In certain embodiments, at least a portion of the PSS exiting growth chamber 120 is analyzed for contaminant(s) such as, without limitation, bacteria. If the analysis of the PSS detects a contaminant, the flow rate of PSS through growth chamber 120 may be increased to remove or substantially reduce the presence of contaminant(s) to a safe level through the system.

The PSS circuit 500 can include a third container 540. The third container 540 can be a waste container. The third container 540 can be configured to receive the PSS. The third container 540 can be configured to receive the PSS after it exits the growth chamber 120. The third container 540 can be fluidly coupled to the second pump 538. A fourth valve 544 can prevent or allow PSS flow from the second pump 538 to the third container 540. In some examples, the fourth valve 544 is manually operable. In other examples, the fourth valve 544 is coupled to the controller. The controller can be configured to send a signal to the valve to open or close the fourth valve 544. The fourth valve 544 can be configured to allow the PSS to flow through the fourth valve 544 when the fourth valve 544 is open. The fourth valve 544 can prevent the flow when the fourth valve 544 is closed.

The PSS circuit 500 can include a fourth container 542. The fourth container 542 can be a waste container. In other examples, the PSS can be received in the fourth container 542 and the fourth container can be removed and coupled to the first valve 504 such that the PSS can be recycled through the PSS circuit 500. In other examples, the fourth container 542 is a drain. The drain can be coupled to a sewer system. The fourth container 542 can be configured to receive the PSS. The fourth container 542 can be fluidly coupled to the second pump 538. A fifth valve 546 can prevent or allow PSS flow from the second pump 538 to the fourth container 542. In some examples, the fifth valve 546 is manually operable. In other examples, the fifth valve 546 is coupled to the controller. The controller can be configured to send a signal to the valve to open or close the fifth valve 546. The fifth valve 546 can be configured to allow the PSS to flow through the fifth valve 546 when the fifth valve 546 is open. The fifth valve 546 can prevent the flow when the fifth valve 546 is closed.

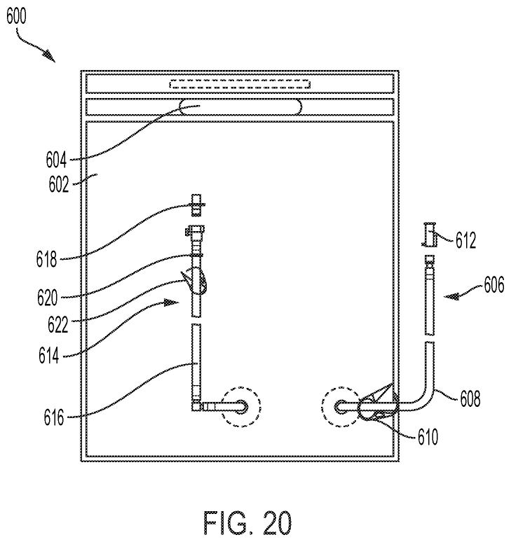

Referring to FIG. 20, at least one of the first, second, third, and fourth containers 502, 508, 540, and 542 can be a receptacle 600. The receptacle 600 can include an outer wall 602 defining an internal cavity. The receptacle 600 can be a bag. The PSS can be stored within the internal cavity. The receptacle 600 can include a mating element 604. The mating element 604 can be configured to mate with a corresponding mating element on the fetal chamber 10 such that the receptacle 600 couples to the fetal chamber 10. The mating element 604 can be an opening that receives a protrusion. In other examples, the mating element 604 can be a hook, protrusion, or adhesive.

The receptacle 600 can include an inlet 606. The PSS can be introduced through the inlet 606 and into the internal cavity. The inlet 606 can include a conduit 608 coupled to the body 602 such that the PSS can flow through the conduit 608 and into the internal cavity. The inlet 606 can include a stopper 610 configured to prevent the flow of PSS through the conduit 608. The stopper 610 can be a clamp. The inlet 606 can include a cap 612. The cap 612 can be detachably coupled to the conduit 608. The cap 612 can prevent PSS flow through the conduit 608 when the cap 612 is coupled to the conduit 608. The conduit 608 can be configured to detachably coupled to a PSS source to introduce the PSS into the internal cavity. In some examples, the conduit 608 is fluidly coupled to one of the fourth and fifth valves 544, 546 of the PSS circuit 500.

The receptacle 600 can include an outlet 614. The PSS can exit the internal cavity through the outlet 614. The outlet 614 can include an outlet conduit 616. The outlet conduit 616 can be coupled to the body 602 such that the PSS can flow through the outlet conduit 616 from the internal cavity. The outlet conduit 616 can be configured to couple to one of the first and second valves 504, 510 of the PSS circuit 500. The outlet conduit 616 can be configured to detachably couple to the PSS circuit 500. In some examples, PSS can be moved from the receptacle 600 into the growth chamber 120 without diluting the PSS. The outlet 614 can include a cap 618. The cap 618 can be detachably coupled to the outlet conduit 616. The cap 618 can prevent PSS flow through the outlet conduit 616 when the cap 618 is coupled to the outlet conduit 616. The outlet 614 can include a seal 620 to form a fluid tight seal between the outlet conduit 616 and the PSS circuit 500. The seal 620 can be an O-ring. The outlet 614 can include an outlet stopper 622. The outlet stopper 622 can be a clamp. The outlet stopper 622 can be detachably coupled to the outlet conduit 616. Each of the cap 618 and the outlet stopper 622 can prevent PSS flow through the outlet conduit 616.

While systems, methods, and compositions have been described in connection with the various embodiments of the various figures, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

What is claimed:

1. A physiologic saline solution comprising:
an aqueous solvent;
from about 1.0 mM to about 2.0 mM calcium chloride;
from about 3.0 mM to about 5.0 mM potassium chloride;
from about 15.0 mM to about 20 mM sodium bicarbonate;
from about 90 mM to about 110 mM sodium chloride; and
between about 9 to about 13 mM sodium acetate,
wherein the solution has a pH ranging from about 7.0 to about 7.4.

2. The solution of claim 1 having an osmolarity ranging from about 250 to about 270 mOsm.

3. The solution of claim 1, wherein the solution further comprises at least one additive selected from a growth factor, an antimicrobial peptide, and combinations thereof.

4. The solution of claim 3, wherein the growth factor is selected from the group consisting of insulin-like growth factor I, insulin-like growth factor II, epidermal growth factor, hepatocyte growth factor, transforming growth factor alpha and transforming growth factor beta-1, and combinations thereof.

5. The solution of claim 4, wherein the antimicrobial peptide is selected from the group consisting of human alpha defensins 1-3, human β-defensin-1, human β-defensin-2, human β-defensin-3, human β-defensin-4, bactericidal/permeability-increasing protein, lactoferrin, cathelicidin, calprotectin, and combinations thereof.

6. The solution of claim 1, further comprising a buffering agent.

7. A gastight container having an interior volume comprising the solution of claim 1.

8. A method of preparing a physiological saline solution, comprising:
dissolving sodium chloride in an aqueous solvent;
dissolving sodium bicarbonate in the aqueous solvent;
dissolving potassium chloride in the aqueous solvent;
dissolving calcium chloride in the aqueous solvent; and
adding a pH-modifying substance comprising sodium acetate to the aqueous solvent in an amount sufficient to adjust the pH to a value of from about 7.0 to about 7.4 to provide the physiological saline solution.

9. The method of claim 8, further comprising the step of introducing an additive selected from a growth factor, an antimicrobial peptide, or a combination thereof to the aqueous solvent.

10. The method of claim 9, wherein the growth factor is selected from the group consisting of insulin-like growth factor I, insulin-like growth factor II, epidermal growth factor hepatocyte growth factor, and combinations thereof.

11. The method of claim 9, wherein the antimicrobial peptide is selected from the group consisting of human β-defensin-1, human β-defensin-2, human β-defensin-3, human (3-defensin-4, bactericidal/permeability-increasing protein, calprotectin, and combinations thereof.

12. The method of claim 8, wherein the solution comprises:
about 1.0 mM to about 2.0 mM calcium chloride;
about 3.0 mM to about 5.0 mM potassium chloride;
about 15.0 mM to about 20 mM sodium bicarbonate;
about 90 mM to about 110 mM sodium chloride; and
about 9 to about 13 mM sodium acetate.

13. The method of claim 8 wherein the solution has an osmolarity ranging from about 250 to about 270 mOsm.

14. The method of claim 8, wherein the step of adding a pH modifying substance further comprises adding hydrochloric acid to the aqueous solvent.

15. The method of claim 8, further comprising:
storing the solution in a gastight vessel.

16. A physiological saline solution circulation system comprising:
a physiological saline solution supply;
a conduit configured to fluidly couple the physiological saline solution supply to a fetal chamber;
a pump coupled to the conduit, the pump configured to pump the physiological saline solution from the physiological saline solution supply to the fetal chamber; and
a filter coupled to the conduit, the filter configured to filter the physiological saline solution as the physiological saline solution is pumped from the physiological saline solution supply to the fetal chamber.

17. The physiological saline solution circulation system of claim 16, wherein the filter is a first filter and the system further comprises:
a second filter fluidly coupled to the conduit, each of the first and second filters configured to filter the physiological saline solution as the physiological saline solution flows from the supply toward the fetal chamber.

18. The physiological saline solution circulation system of claim 17, wherein one of the first and second filters are configured to be replaced while the physiological saline solution flows through the other of the first and second filters.

19. The physiological saline solution circulation system of claim 16 further comprising a pressure sensor configured to sense pressure within the conduit.

20. The physiological saline solution circulation system of claim 16, further comprising a disinfector configured to disinfect the physiological saline solution.

21. The physiological saline solution circulation system of claim 20, wherein the disinfector is a first disinfector and is configured to be positioned upstream from the fetal chamber, and
wherein the system includes a second disinfector configured to be positioned downstream from the fetal chamber, the second disinfector configured to disinfect the physiological saline solution.

22. The physiological saline solution circulation system of claim 20, wherein the disinfector comprises an ultraviolet light source.

23. The physiological saline solution circulation system of claim 22, wherein the conduit is elongate along a conduit central axis and the disinfector is configured to emit ultraviolet light along a light central axis that is disposed at an angle relative to the conduit central axis so as to prevent the ultraviolet light from moving through the conduit into the fetal chamber.

24. The physiological saline solution circulation system of claim 23, wherein the angle is about 90 degrees.

25. The physiological saline solution circulation system of claim 20, wherein the disinfector is configured to emit radio frequency waves to disinfect the physiological saline solution.

26. The physiological saline solution circulation system of claim 20, wherein the disinfector is configured to emit radio frequency waves to heat the physiological saline solution.

27. The physiological saline solution circulation system of claim 16, further comprising:
a heat exchanger configured to heat the physiological saline solution.

28. The physiological saline solution circulation system of claim 16, further comprising:
a pressure relief valve coupled to at least one of the fetal chamber, growth chamber, and combinations thereof.

29. The physiological saline solution circulation system of claim 16, wherein the pump is a first pump and the system further comprises:
a second pump coupled to the conduit, the second pump configured to pump the physiological saline solution from fetal chamber to a waste container.

30. The physiological saline solution circulation system of claim 29, wherein the first pump is positioned upstream of the fetal chamber and the second pump is positioned downstream of the fetal chamber.

31. The physiological saline solution circulation system of claim 16, further comprising a temperature sensor coupled to the conduit, the temperature sensor configured to sense a temperature of the physiological saline solution.
